(19) United States
(12) Patent Application Publication
(10) Pub. No.: US 2015/0250822 A1
(43) Pub. Date: Sep. 10, 2015

(54) PLATELET COMPOSITIONS AND USES THEREOF

(71) Applicants: Giannoula Lakka Klement, Boston, MA (US); Abdo Abou-Slaybi, Brighton, MA (US); Nandita Bhattacharya, Brighton, MA (US)

(72) Inventors: Giannoula Lakka Klement, Boston, MA (US); Abdo Abou-Slaybi, Brighton, MA (US); Nandita Bhattacharya, Brighton, MA (US)

(73) Assignee: GeneSys Research Institute, Brighton, MA (US)

(21) Appl. No.: 14/433,001

(22) PCT Filed: Oct. 4, 2013
(86) PCT No.: PCT/US2013/063579

§ 371 Date: Apr. 1, 2015

Related U.S. Application Data
(60) Provisional application No. 61/709,859, filed on Oct. 4, 2012.

(51) Int. Cl. A61K 35/19; A61K 45/06; A61K 38/18

(52) U.S. Cl. CPC A61K 35/19; A61K 38/1858; A61K 38/1825; A61K 38/1866; A61K 45/06

(57) ABSTRACT

The invention generally features isolated platelets, compositions, methods, and kits useful for targeted delivery of one or more therapeutic agents to a site of injury, inflammation, disease, or disorder. Also featured are methods and kits for producing a platelet loaded with one or more therapeutic agents. Platelets loaded with one or more therapeutic agents are useful for treating neoplasia, hemophilia, wounds, and other pathologies or conditions involving sites of injury, inflammation, disease, or disorder where platelets are able to localize.

### CROSS-REFERENCE TO RELATED APPLICATIONS

This application is claims priority from U.S. Provisional Patent Application Ser. No. 61/709,859, filed Oct. 4, 2012, which is incorporated by reference herein in its entirety.

### STATEMENT OF RIGHTS TO INVENTIONS MADE UNDER FEDERALLY SPONSORED RESEARCH

This work was supported by the following grant from the National Institutes of Health, Grant No.: 1R01GM093050-01A1. The government has certain rights in the invention.

### BACKGROUND OF THE INVENTION

Sites of injury, inflammation, and/or angiogenesis, are characteristics of pathological situations such as wound healing, tumors, atherosclerotic plague or neural degeneration and physiological events such as embryo implantation or tissue regeneration. Synthetic homing mechanisms, such as peptides targeting tumor vasculature, have not been able to achieve specificity for targeting therapeutic agents to such sites. Effective delivery of agents to these sites has been an obstacle to developing therapies for angiogenesis related diseases and conditions.

Angiogenesis, the process of developing a novel vascular network from a pre-existing one, is tightly controlled by various endogenous regulators. These regulatory elements include both pro- and anti-angiogenic proteins that finely modulate the neovascular morphological and functional characteristics. Where the regulation of such processes is disrupted, inappropriate angiogenesis can have severe consequences. For example, solid tumors, which are vascularized as a result of angiogenesis, have the potential to rapidly grow and metastasize.

An urgent need exists for treatment of angiogenesis related diseases and conditions. Accordingly, new methods of targeting sites of injury, inflammation, and/or angiogenesis are urgently required.

### SUMMARY OF THE INVENTION

As described below, the present invention generally features angiogenesis modulators, related prophylactic and therapeutic methods, as well as screening methods for the identification of such agents.

In one aspect, the invention provides a method for delivering an agent to a site of injury, inflammation, disease, or disorder in a subject in need thereof, the method comprising loading one or more samples of platelets with one or more agents, each agent comprising a heparin binding domain, thereby producing one or more pools of loaded platelets; administering a composition comprising one or more pools of loaded platelets to the subject in need thereof, wherein an individual loaded platelet present in the composition localizes to a site of injury, inflammation, disease, or disorder and delivers its one or more agents to the site of injury, inflammation, disease, or disorder.

In another aspect, the invention provides a method for preparing a platelet loaded with one or more agents, the method comprising obtaining a platelet, contacting the platelet in vitro with the one or more agents, each agent comprising a heparin binding domain, allowing contact between the platelet and the one or more agents to progress until the one or more agents is internalized by the platelet, thereby producing a loaded platelet.

In still another aspect, the invention provides an isolated platelet loaded with one or more agents in vitro, wherein the agent comprises a heparin binding domain.

In a related aspect, the invention provides a composition or pharmaceutical composition containing a loaded platelet of the invention. The pharmaceutical composition contains an effective amount of the loaded platelet of the invention in a pharmaceutically acceptable excipient.

In an additional aspect, the invention provides a kit for treating an injury, inflammation, disease, or disorder, the kit comprising the loaded platelet, composition, or pharmaceutical composition. The kit may further comprise written instructions for using the platelet for the treatment of a subject.

In yet another aspect, the invention provides a kit for preparing a loaded platelet of the present invention. The kit may further comprise written instructions for preparing the loaded platelet and/or using the loaded platelet for the treatment of a subject.

In various embodiments of any of the aspects delineated herein, the heparin binding domain is operably linked to the one or more agents. In various embodiments, the one or more agents is a recombinant fusion polypeptide. In various embodiments of any of the aspects delineated herein, the loaded platelet is obtained from platelet rich plasma. In certain embodiments, the platelet is autologous or heterologous.

In various embodiments of any of the aspects delineated herein, the one or more agents is a growth factor, a growth inhibitor, a protease/proteinase, a coagulation factor, a lipid or phospholipid, an extracellular matrix protein, a hormone, an enzyme, a chemokine/chemoattractant, or a neurotrophin Examples of a growth factor include vascular endothelial growth factor (VEGF), basic fibroblast growth factor (bFGF), and platelet-derived growth factor (PDGF), Epidermal Growth Factor (EGF), Hepatocyte Growth Factor (HGF), Insulin-Like Growth Factor (IGF), and an Angiopoietin. Examples of growth inhibitor include angiostatin, endostatin, tumstatin, Thrombospondin-1 (TSP1), Platelet Factor 4 (PF4, CXCL4), and Tissue Inhibitors of Metalloproteinases (TIMPs). Examples of a protease/proteinase include Matrix Metalloproteinases (MMPs), thrombin, tissue plasminogen activator (tPA), urokinase, and streptokinase. Examples of a coagulation factor include Factor II (thrombin), Antithrombin III (ATIII), Kallikrein, tissue factor (TF), Factor V, Factor VII, Factor VIII, Factor IX, Factor X, Factor XI, and Factor XII, Factor XIII, Fibrinogen, Protein S, Protein C, thrombomodulin, plasminogen, and tissue factor pathway inhibitor (TFPI). Examples of a lipid or phospholipid include apolipoprotein E (ApoE), platelet phospholipids, and Sphingosine-1-phosphate (S1P). Examples of an extracellular matrix protein include integrins, fibronectin, laminin, focal adhesion proteins (FAK), vinculin, talin, actin filaments, and collagen. Examples of a hormone include insulin, steroid, erythropoietin, thrombopoietin, and thyroid hormone. Examples of an enzyme are Heparanase and a Matrix Metalloproteinase (MMP). Examples of a chemokine/chemoattractant include Connective Tissue Growth Factor (CTGF), Stromal Cell-derived Factor-1 (SDF-1) (CXCL12), interleukins (IL1, 2, 6, 8), and CD40 Ligand (CD40L, CD154). Examples of a neurotrophin include nerve growth factor (NGF), brain-derived neurotrophic factor (BDNF), Neurotrophin-3 (NT-3), and Neurotrophin 4/5 (NT-4/5).

In various embodiments of any of the aspects delineated herein, the one or more agents is a cytotoxic compound, a small molecule, an antibody, or a factor that inhibits angiogenesis.

In various embodiments of any of the aspects delineated herein, a loaded platelet comprises 1 to 1000 fold more copies of the one or more agents than the platelet comprised prior to loading the one or more agents. The amount of cargo loaded may be a small amount (i.e., the same amount as normally present or less than that normally present in a platelet) or a large amount (i.e., 2 to 1000 times as much as normally present in a platelet, e.g., up to two times, five times, ten times, twenty-five, fifty times, 100 times, 200 times, 300 times, 400 times, 500 times, 600 times, 700 times, 800 times, 900 times, and 1000 times).

In various embodiments of any of the aspects delineated herein, the site of injury, inflammation, disease, or disorder includes wound, tumor, atherosclerotic plaque, macular degeneration, skin proliferation/ulceration (psoriasis, rosacea), gastrointestinal proliferation/ulceration (ulcerative colitis, Crohn's disease), arthritis and joint disease (synovial plague), endometriosis, site of neural degeneration (Alzheimer Disease, ALS, MS), post infectious angiogenesis (Bartonella, shigelosis, cerebral malaria), embryo implantation, preeclampsia, obesity, neuropathy, and tissue regeneration.

In various embodiments of any of the aspects delineated herein, the agent is a growth factor, coagulation factor, or neurotrophin In various embodiments, the growth factor is one or more of vascular endothelial growth factor (VEGF), basic fibroblast growth factor (bFGF), and platelet-derived growth factor (PDGF). In particular embodiments, the coagulation factor is one or more of Factor VIII, Factor IX, Factor XI, and Factor XII. In certain embodiments, the neurotrophin is selected from the group consisting of nerve growth factor (NGF), brain-derived neurotrophic factor (BDNF), Neurotrophin-3 (NT-3), and Neurotrophin 4/5 (NT-4/5). In various embodiments of any of the aspects delineated herein, the agent is a cytotoxic compound or a factor that inhibits angiogenesis. In various embodiments of any of the aspects delineated herein, method further comprising administering one or more of heparanase, PAR1 agonist peptide, or PAR4 agonist peptide to enhance release of said agent from the loaded platelet.

In various embodiments of any of the aspects delineated herein, the method comprises administering a plurality of sequential doses of compositions. In various embodiments, the plurality of sequential doses of compositions comprises compositions each including platelets loaded with identical one or more agents. In various embodiments, the loaded platelets in a composition include a lesser amount, the same amount, or a greater amount of the identical one or more agents than the amount loaded into platelets in another composition. In various embodiments, the plurality of sequential doses of compositions comprises a variety of compositions. In various embodiments, loaded platelets in a composition include identical one or more agents or different one or more agents than agents loaded into platelets in another composition. In various embodiments, loaded platelets in a composition include identical one or more agents than agents loaded in another composition, the loaded platelets in the composition include a lesser amount, the same amount, or a greater amount of the identical one or more agents than the amount loaded into platelets in the other composition. In various embodiments, a specific composition administered to the subject is selected based upon the particular injury, inflammation, disease, or disorder afflicting the subject. In various embodiments, a specific composition administered to the subject is selected based upon the stage or severity of the particular injury, inflammation, disease, or disorder afflicting the subject. In various embodiments, a specific composition administered to the subject is selected based upon characteristics of the subject including but not limited to the subject's response to a previously administered composition if a composition had been previously administered.

In various embodiments of any of the aspects delineated herein, method further comprising administering one or more of heparanase, PAR1 agonist peptide, and/or PAR4 agonist peptide to enhance release of said agent from the platelet.

Any loaded platelet, composition, or method provided herein can be combined with one or more of any of the other isolated platelets, compositions, and methods provided herein.

Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof. An “agent” all isoforms of said agent.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “alteration” is meant a change in the sequence or in a modification (e.g., a post-translational modification) of a gene or polypeptide relative to an endogenous wild-type reference sequence.

By “ameliorate” is meant decrease, suppress, attenuate, diminish, arrest, or stabilize the development or progression of a disease.

By “antibody” is meant any immunoglobulin polypeptide, or fragment thereof, having immunogen binding ability.

By “thrombin receptor” is meant a transmembrane G protein-coupled family of receptors activated by protease (e.g., thrombin) on the cell surface of platelets. Exemplary thrombin receptors include PAR1, which regulates the release of angiogenesis stimulants from platelets, and PAR4, which regulates the release of angiogenesis inhibitors from platelets.

By “autologous” is meant to refer to the transplantation of cells, tissues, organs, or even proteins obtained from an individual to the same individual.

By “blood vessel formation” is meant the dynamic process that includes one or more steps of blood vessel development and/or maturation, such as angiogenesis, vasculogenesis, formation of an immature blood vessel network, blood vessel remodeling, blood vessel stabilization, blood vessel maturation, blood vessel differentiation, or establishment of a functional blood vessel network.

By “angiogenesis” is meant the growth of new blood vessels originating from existing blood vessels. Angiogenesis can be assayed by measuring the total length of blood vessel segments per unit area, the functional vascular density (total length of perfused blood vessel per unit area), or the vessel volume density (total of blood vessel volume per unit volume of tissue).

By “vasculogenesis” is meant the development of new blood vessels originating from stem cells, angioblasts, or other precursor cells.

By “blood vessel stability” is meant the maintenance of a blood vessel network.

By “coagulation factor” is meant a polypeptide, analog, or fragment thereof having at least 85, 90, 95, 96, 97, 98, 99 or 100% amino acid sequence identity to a member of the proteins involved in the coagulation cascade, by which blood clots are formed. Exemplary coagulation factors include Factor VIII, Factor IX, Factor XI, and Factor XII. Disorders of coagulation include hemophilia, which is due to a deficiency of Factor VIII or Factor IX.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “alteration” is meant a change in the sequence or in a modification (e.g., a post-translational modification) of a gene or polypeptide relative to an endogenous wild-type reference sequence.

By “ameliorate” is meant decrease, suppress, attenuate, diminish, arrest, or stabilize the development or progression of a disease.

By “antibody” is meant any immunoglobulin polypeptide, or fragment thereof, having immunogen binding ability. By “thrombin receptor” is meant a transmembrane G protein-coupled family of receptors activated by protease (e.g., thrombin) on the cell surface of platelets. Exemplary, thrombin receptors include PAR1, which regulates the release of angiogenesis stimulants from platelets, and PAR4, which regulates the release of angiogenesis inhibitors from platelets.

By “compound” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

A “cancer” in an animal refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells.

By “disease” is meant any condition or disorder that damages or interferes with the normal function of a cell, tissue, or organ.

By “an effective amount” is meant the amount required to ameliorate the symptoms of a disease relative to an untreated patient. The effective amount of active compound(s) used to practice the present invention for therapeutic treatment of an angiogenesis-associated disease varies depending upon the manner of administration, the age, body weight, and general health of the subject. Ultimately, the attending physician or veterinarian will decide the appropriate amount and dosage regimen. Such amount is referred to as an “effective” amount.

By “exogenous” is meant a nucleic acid molecule or polypeptide that is not endogenously present in the cell, or not present at a level sufficient to achieve the functional effects obtained when over-expressed. The term “exogenous” would therefore encompass any recombinant nucleic acid molecule or polypeptide expressed in a cell, such as foreign, heterologous, and over-expressed nucleic acid molecules and polypeptides.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Fusion polypeptide” or “fusion protein”, as used herein, is meant a polypeptide comprising two or more different polypeptides or active fragments thereof that are not naturally present in the same polypeptide. Generally, the two or more different polypeptides are linked together covalently, e.g., chemically linked or fused in frame by a peptide bond.

By “growth factor” is meant a polypeptide, analog, or fragment thereof having an activity capable of stimulating cellular growth, proliferation, and/or cellular differentiation, including vascular endothelial growth factor (VEGF), basic fibroblast growth factor (bFGF), and platelet-derived growth factor (PDGF). A growth factor has at least 85, 90, 95, 96, 97, 98, 99 or 100% amino acid sequence identity to any of these proteins and has an activity that induces cellular growth, proliferation, and/or cellular differentiation.

By “heparin” is meant a highly sulfated glycosaminoglycan polymer consisting of a variably sulfated repeating disaccharide unit. Heparin is used as an anticoagulant

By “heparin binding domain” is meant a linear or spatial domain in a protein that binds heparin through electrostatic interactions. Exemplary linear heparin binding domains include two consensus sequences of amino acids: XBBXBX and XBBBXXBX, where B is a basic residue (e.g., arginine and lysine) and X is a hydropathic residue. Methods for determining heparin binding are known in the art, including use of heparin or heparan sulfate conjugated media to detect protein binding in the presence of increasing salt concentration.

By “heparinase” is meant an enzyme that cleaves polysaccharides containing 1,4-linked D-glucuronate or L-iduronate residues and 1,4-alpha-linked 2-sulfoamino-2-deoxy-6-sulfo-D-glucose residues (e.g., heparin) to yield oligosaccharides with terminal 4-deoxy-alpha-D-gluc-4-enuronosyl groups at their non-reducing ends.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell (e.g., a platelet) that is separated from the molecular and/or cellular components that naturally accompany the cell.

By “isolated nucleic acid molecule” is meant a nucleic acid (e.g., a DNA) that is free of the genes, which, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “ligand” as used herein refers to a molecule that binds to a receptor.

“Linker”, as used herein, is meant a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple a heparin binding domain to a polypeptide).

By “marker” is meant any protein or polynucleotide having an alteration in expression level or activity that is associated with a disease or disorder.

By “modulate” is meant positively or negatively alter. Exemplary modulations include a 1%, 2%, 5%, 10%, 25%, 50%, 75%, or 100% change.

“By “neoplasia” is meant a disease that is caused by or results in inappropriately high levels of cell division, inappropriately low levels of apoptosis, or both. Neoplasia is characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells). Illustrative neoplasms for which the invention can be used include, but are not limited to leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

By “neurotrophin” is meant a polypeptide, analog, or fragment thereof that induce the survival, development, and/or function of neurons, including nerve growth factor (NGF), brain-derived neurotrophic factor (BDNF), Neurotrophin-3 (NT-3), and Neurotrophin 4/5 (NT-4/5). A neurotrophin has at least 85, 90, 95, 96, 97, 98, 99 or 100% amino acid sequence identity to any of these proteins and has an activity that promotes or maintains neuronal function.

The term “obtaining” as in “obtaining the agent” is intended to include purchasing, synthesizing or otherwise acquiring the agent (or indicated substance or material).

By “operably linked” is meant the linking of two or more biomolecules so that the biological functions, activities, and/or structure associated with the biomolecules are at least retained. In reference to polypeptides, the term means that the linking of two or more polypeptides results in a fusion polypeptide that retains at least some of the respective individual activities of each polypeptide component. The two or more polypeptides may be linked directly or via a linker. In reference to nucleic acids, the term means that a first polynucleotide is positioned adjacent to a second polynucleotide that directs transcription of the first polynucleotide when appropriate molecules (e.g., transcriptional activator proteins) are bound to the second polynucleotide.

By “peptide” is meant any fragment of a polypeptide. Typically peptide lengths vary between 5 and 1000 amino acids (e.g., 5, 10, 15, 20, 25, 50, 100, 200, 250, 500, 750, and 1000).

By “platelet” is meant a non-nucleated disk-shaped cell formed in the megakaryocyte and found in the blood of all mammals. Platelets are frequently involved in blood coagulation.

By “polypeptide” is meant any chain of amino acids, regardless of length or post-translational modification.

By “promoter” is meant a polynucleotide sufficient to direct transcription.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

By “receptor” is meant a polypeptide, or portion thereof, present on a cell membrane that selectively binds one or more ligand.

By “reduce” or “increase” is meant to alter negatively or positively, respectively, by at least 5%. An alteration may be by 5%, 10%, 25%, 30%, 50%, 75%, or even by 100%.

By “reference” is meant a standard or control condition.

By “subject” is meant a mammal, including, but not limited to, a human or non-human mammal, such as a bovine, rodent, equine, canine, ovine, or feline.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and even more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

“Sequence identity” or “identity” in the context of two nucleic acid or polypeptide sequences includes reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not deleteriously change the functional properties of the molecule. Where sequences differ in conservative substitutions, the percent sequence identity may be adjusted upwards to correct for the conservative nature of the substitution. Sequences which differ by such conservative substitutions are said to have sequence similarity. Approaches for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers and Miller, Computer Applic. Biol. Sci., 4: 11-17, 1988, for example, as implemented in the program PC/GENE (Intelligenetics, Mountain View, Calif., USA).

“Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “homologous” in their various grammatical forms in the context of polynucleotides means that a polynucleotide comprises a sequence that has a desired identity, for example, at least 60% identity, preferably at least 70% sequence identity, more preferably at least 80%, still more preferably at least 90% and even more preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, more preferably at least 70%, 80%, 85%, 90%, and even more preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, for example, when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid, although such cross-reactivity is not required for two polypeptides to be deemed substantially identical.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

A “recombinant host” may be any prokaryotic or eukaryotic cell that contains either a cloning vector or expression vector. This term also includes those prokaryotic or eukaryotic cells that have been genetically engineered to contain the cloned gene(s) in the chromosome or genome of the host cell.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 5, 10, or 15 amino acids, preferably at least about 20 amino acids, more preferably at least about 25 amino acids, and even more preferably about 35 amino acids, about 50 amino acids, about 100 amino acids, or about 150 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides about 300 nucleotides or about 450 nucleotides or any integer thereabout or therebetween.

Methods of alignment of sequences for comparison are well-known in the art. Optimal alignment of sequences for comparison may be conducted by the local homology algorithm of Smith and Waterman, Adv. Appl. Math., 2: 482, 1981; by the homology alignment algorithm of Needleman and Wunsch, J. Mol. Biol., 48: 443, 1970; by the search for similarity method of Pearson and Lipman, Proc. Natl. Acad. Sci. USA, 8: 2444, 1988; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, Gene, 73: 237-244, 1988; Corpet, et al., Nucleic Acids Research, 16:881-90, 1988; Huang, et al., Computer Applications in the Biosciences, 8:1-6, 1992; and Pearson, et al., Methods in Molecular Biology, 24:7-331, 1994. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and can be used with the present invention.

Unless otherwise stated, sequence identity/similarity values provided herein refer to the value obtained using the BLAST 2.0 suite of programs, or their successors, using default parameters (Altschul et al., Nucleic Acids Res, 2:3389-3402, 1997). It is to be understood that default settings of these parameters can be readily changed as needed in the future.

As those ordinary skilled in the art will understand, BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163, 1993) and XNU (Clayerie and States, Comput. Chem., 17:191-1, 1993) low-complexity filters can be employed alone or in combination.

The term “subject” as used herein refers to a vertebrate, preferably a mammal, more preferably a human. A subject is typically one who is in need of a treatment.

By “thrombin receptor” is meant a transmembrane G protein-coupled family of receptors activated by protease (e.g., thrombin) on the cell surface of platelets. Exemplary, thrombin receptors include PAR1, which regulates the release of angiogenesis stimulants from platelets, and PAR4, which regulates the release of angiogenesis inhibitors from platelets.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

A “tumor,” as used herein, refers to all neoplastic cell growth and proliferation, whether malignant or benign, and all precancerous and cancerous cells and tissues.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Unless specifically stated or obvious from context, as used herein, the term “about” is understood as within a range of normal tolerance in the art, for example within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Unless specifically stated or obvious from context, as used herein, the term “or” is understood to be inclusive. Unless specifically stated or obvious from context, as used herein, the terms “a”, “an”, and “the” are understood to be singular or plural.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other aspects of the invention are described in the following disclosure and are within the ambit of the invention.

### BRIEF DESCRIPTION OF THE DRAWINGS

The following Detailed Description, given by way of example, but not intended to limit the invention to specific embodiments described, may be understood in conjunction with the accompanying drawings.

FIG. 1 depicts human resting platelets selectively take up PDGF.

FIG. 2 depicts sequestration of growth factors and chemokines into platelets. The difference between the gray & black bars reflects internal fraction. *p<0.05 T-Test; n=3

FIG. 3 depicts inhibition of individual receptors had no effect on platelet sequestration of angiogenesis regulators.

FIG. 4 depicts that surfen, an inhibitor of heparan sulfate binding, prevented platelet sequestration of angiogenesis regulators.

FIG. 5 depicts that surfen blocked uptake of angiogenesis regulators, but not of TPO.

FIG. 6 depicts a number of VEGF isoforms created by alternative splicing of the gene have different functions and properties, including heparin binding affinity.

FIG. 7 depicts elution of angiogenesis regulators from platelet clot on a heparin Column.

FIG. 8 depicts the concentration of angiogenesis inhibitors in platelets surpass many fold the concentration of stimulators.

FIG. 9 depicts sequential release of angiogenesis regulators from platelets.

FIG. 10 depicts internalized fluorescent staining of VEGF₁₆₅ in Human Microvascular Endothelial Cells (HMVEC) cells.

FIG. 11 depicts internalized fluorescent staining of VEGF₁₆₄ in HMVEC cells.

FIG. 12 depicts a lack of depicts internalized fluorescent staining of VEGF₁₂₁ isoforms in HMVEC cells.

FIG. 13 depicts a differential effect of platelets on proliferation of a Human neuroblastoma cell line (SK-N-SH) and endothelial (HMVEC) cells.

FIG. 14 depicts individual proteins did not differentially regulate growth in HMVEC or SK-N-SH.

FIG. 15 depicts individual inhibitors had the same effects on HMVEC and SK-N-SH

FIG. 16 depicts apoptosis arrays of platelet treated SK-N-SH compared to untreated controls.

FIG. 17 depicts platelet treated SK-N-SH showed increased apoptosis when compared to untreated controls.

FIG. 18 depicts platelet mediated inhibitory effects were found in other tumor types

FIG. 19 depicts heparin rescued SK-N-SH neuroblastoma from platelet-mediated apoptosis, but not from platelet-mediated HMVEC proliferation

FIG. 20 depicts infusion of 200 μL of human Platelet Rich Plasma (PRP) Q3 days improved the survival of mice with disseminated orthotopic SK-N-SH neuroblastoma.

FIG. 21 depicts examples of loaded platelets, of compositions, and of treatment doses, and an exemplary treatment regimen.

### DETAILED DESCRIPTION OF THE INVENTION

The invention generally features compositions and methods that are useful for targeted delivery of agents (e.g., therapeutic compounds or proteins) to sites of injury, inflammation, and angiogenesis, as well as, methods that provide for the loading of such agents into platelets.

It has been described that platelets can selectively and actively (i.e., against a concentration gradient) sequester angiogenesis, growth and inflammation regulating proteins. However, the mechanisms of this selective and active process were unclear. As described in more detail below, the present invention is based on the discovery that proteins are taken up by platelets based on their ability to bind heparin. Contrary to the commonly held opinion that angiogenesis regulators (e.g., VEGF, bFGF, PDGF, PF4, HGF), are taken up by platelets in a receptor mediated process, it has been found that no receptor engagement actually occurs and the inhibition of the respective receptor does not inhibit the uptake.

Thus, the present inventors have appreciated that addition of a heparin-binding (or heparan sulfate-binding) domain to a substance can facilitate the “loading” of various substances into platelets. Because platelets migrate and localize to areas of acute inflammation, this discovery is widely applicable to a variety of pathological conditions including but not limited to wound healing, anti-cancer therapy (e.g., by delivering therapeutic agents to tumors), atherosclerotic plaques, neural degeneration, as well as to physiological events, such as embryo implantation or tissue regeneration.

The method of using platelets as the mode of delivery for therapeutic agents or growth factors has many advantages over the existing methods. Most synthetic homing mechanisms such as peptides targeting tumor vasculature have not been able to achieve the specificity that platelets are able to achieve. The ability to use autologous platelets adds an additional feature of safety to the procedure, and the speed of “loading” (seconds to minutes for proteins that contain a heparin-binding domain) indicates that use of platelets loaded with therapeutic agents is readily and easily translated to clinics and applied in a clinical setting. As technology advances, it will likely be possible to translate the invention to a home, an office, or an outpatient setting using portable or semi-portable equipment.

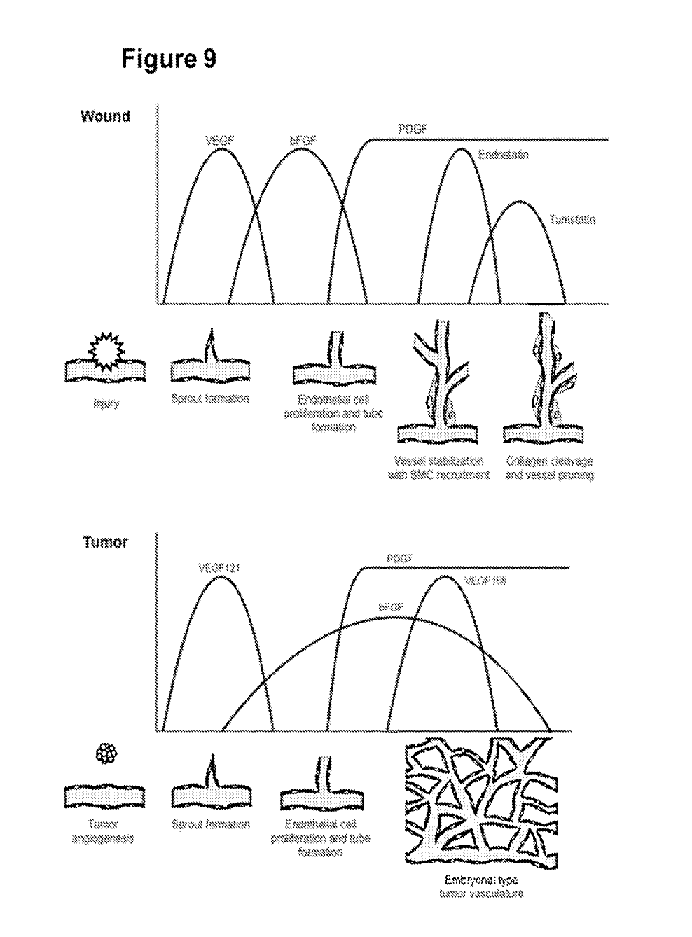

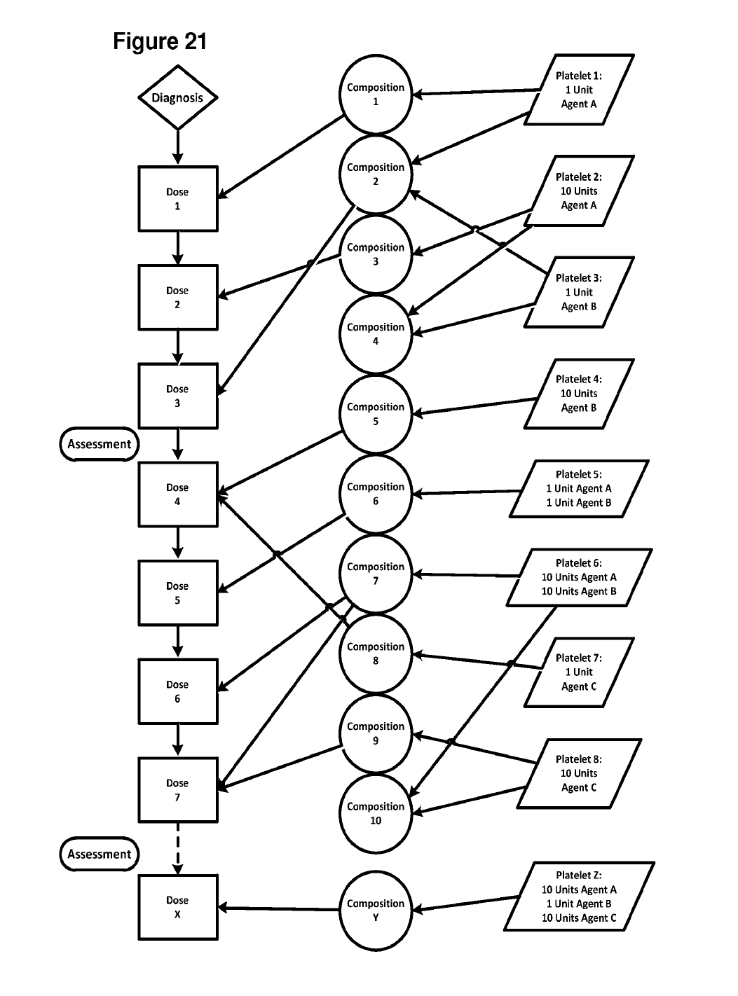

Additionally, it is noteworthy that an individual platelet is capable of holding a large number of copies of its cargo; thus, upon contact with the platelet's target cell, it is able to deliver, in an amplified, highly-concentrated, rapid, and focused manner, the contents of its cargo onto one or a few target cells. Alternately, a platelet can be loaded with a lower quantity of its cargo, thereby allowing it to deliver a lower dose of its cargo, yet still in a rapid and focused manner. Moreover, each platelet is able to contain a variety of different cargo compounds; thus, a platelet can deliver a mixture of cargo compounds. Furthermore, a composition of loaded platelets can include a variety of platelets each containing a different cargo compound and/or different amounts of each cargo type; thus, the milieu of compounds can be delivered according to a subject's need. When sequential doses are administered to a subject, each composition dosed may differ from a prior dose's composition. (See, FIG. 21) A dosing regimen may approximate a naturally occurring process (e.g., wound healing as shown in FIG. 9, top) or may exploit a known naturally occurring process to enact a desired outcome.

### Platelets

Platelets, or thrombocytes, are small, irregularly shaped clear cell fragments (i.e. cells that do not have a nucleus containing DNA), 2-3 μm in diameter, which are derived from fragmentation of precursor megakaryocytes. The function of platelets is the maintenance of hemostasis. This is achieved primarily by the formation of thrombi, when damage to the endothelium of blood vessels occurs. On the converse, thrombus formation must be inhibited at times when there is no damage to the endothelium.

Platelets are recruited to wound and tumor sites and are among the first responders to these sites. Platelets are immediately available as “acute phase reactant.” The average half-life of a platelet is about four to seven days. However, they are continuously replenished or renewed. Platelets attach to sites of injury and create a provisional matrix (a clot) that contains factors for repairing the injury. Such factors include inflammatory cytokines (e.g., Interleukins IL1, IL6, IL8, and TGFβ1); angiogenesis stimulators (e.g., VEGF, bFGF, HGF, PDGF, angiopoietin 1&2, MMP2); angiogenesis inhibitors (e.g., PF4, TSP1, endostatin, tumstatin, angiostatin); tissue repair facilitators (e.g., BDNF, NGF, IGF1, EGF); tissue invasion facilitators (e.g., MMP9, heparinase); and stem cell attractants (e.g., SDF1). Platelets are a natural source of growth factors, which are stored in alpha granules. They release a multitude of growth factors including vascular endothelial growth factor (VEGF), basic fibroblast growth factor (bFGF), platelet-derived growth factor (PDGF), a potent chemotactic agent, and TGF beta, which stimulates the deposition of extracellular matrix. These growth factors have been shown to play a significant role in the repair and regeneration of connective tissues. Other healing-associated growth factors produced by platelets include, insulin-like growth factor 1 (IGF-1) and platelet-derived epidermal growth factor (PDGF).

It has been observed that angiogenesis regulators are sequestered in platelets actively, and against the concentration gradient (Cervi D et al. Blood. 2008 Feb. 1; 111(3):1201-7, Klement G L et al. Blood. 2009 Mar. 19; 113(12):2835-42). Furthermore, angiogenesis regulator sequestration is selective (Klement G L et al. Blood. 2009 Mar. 19; 113(12):2835-42). There exist different fractions of alpha-granules for organizing the storage of angiogenesis regulators (Italiano J E Jr et al, Blood. 2008 Feb. 1; 111(3):1227-33), and there is differential release of functionally distinct groups of proteins, based on high and low affinity thrombin receptors (Ma et al, PNAS, 2005 Jan. 4; 102(1): 216-220, Italiano J E Jr et al, Blood. 2008 Feb. 1; 111(3):1227-33). Angiogenesis during wound healing is facilitated by reciprocal interaction of platelets and stromal cells, and is not stimulated by individual growth factors (Pietramaggiori G. et al, Am J Pathol. 2008 Dececember; 173(6):1609-16).

As shown herein, the discovery that the sequestration of angiogenesis regulators in platelets by their heparin binding affinity is physiologically relevant and accounts for these various observations. It has been found that sequestration of angiogenesis regulators is not dependent on the presence or activation of the respective receptors. The release of angiogenesis regulators is dependent on heparan sulfate binding affinity, as well as on the amount of thrombin in the tissues (PAR1 vs PAR4).

Without being bound to theory, the affinity for heparin creates a biological system which allows for temporal, spatial and environmental controlled release of angiogenesis regulators from platelets. Indeed, VEGF isoforms have different affinities for heparin, which may account for their different functions.

### Heparin and Heparan Sulfate (HS)

Heparin is a linear, polydisperse polysaccharide consisting of repeating units of 1→4-linked pyranosyluronic acid and 2-amino-2-deoxyglucopyranose (glucosamine) residues. The uronic acid residues typically consist of 90% 1-idopyranosyluronic acid (1-iduronic acid) and 10% d-glucopyranosyluronic acid (d-glucuronic acid). Structural variations of this disaccharide exist, leading to heparin microheterogeneity. The amino group of the glucosamine residue may be substituted with an acetyl or sulfo group or unsubstituted. The 3 and 6 positions of the glucosamine residues can either be substituted with an O-sulfo group or unsubstituted. The uronic acid, which can either be 1-iduronic or d-glucuronic acid, may also contain a 2-O-sulfo group. Heparan sulfate (HS) is also a linear copolymer of uronic acid 1→4 linked to glucosamine but has a more varied structure. d-glucuronic acid predominates in HS, although substantial amounts of 1-iduronic acid can be present. Additionally, HS is much less substituted in sulfo groups than heparin.

Heparin is a polydisperse polysaccharide with a heterogeneous saccharide sequence that binds a large number of proteins. Consequently, there is a wide range of possible binding sites along the heparin chain as well as a diversity of heparin-binding sites among the numerous proteins that bind this glycosaminoglycan (GAG). Most heparin-binding proteins also bind heparan sulfate (HS). Whereas heparin is primarily intracellular, the HS proteoglycan (HSPG) is a common constituent of cell surfaces and the extracellular matrix (ECM) and is also involved in a wide range of biological functions. Although heparin and HS are structurally related, subtle differences in their saccharide sequence make it difficult to determine common features in heparin-protein interactions

### Heparin-Binding Domains

Heparin binding domains are useful in the compositions and methods of the invention, as they are used to load platelets with cargo. Heparin binding domains may be chemically conjugated to an agent (i.e., a therapeutic agent) or genetically expressed to produce a fusion protein having a therapeutic polypeptide operably linked to the heparin binding domain; thereby producing a protein-active agent or fusion protein for a protein that may not normally have a heparin binding domain. The activities of both the heparin binding domain and the coupled agent are retained in the resulting molecule.

Heparin-binding sites in proteins are characterized by the presence of clusters of positively charged basic amino acids that form ion pairs with spatially defined negatively charged sulfo or carboxyl groups of the GAG chain. As heparin has an average of 2.7 negative charges per disaccharide (<2 negative charges per disaccharide for HS) provided by sulfo and carboxyl groups, a prominent type of interaction between heparin (or HS) and proteins is ionic. Nonelectrostatic interactions such as hydrogen bonding and hydrophobic interactions can also contribute to the stability of heparin-protein complexes. The highly anionic nature of heparin has led the field to believe that its binding with proteins is nonspecific in nature. However, elucidation of the structure activity relationship of the binding between heparin and its binding proteins has demonstrated that heparin can have defined sequences within its binding domain that interact with high affinity with its target proteins in a specific manner. Furthermore, other sequences have been discovered that interact with some level of specificity to growth factors, growth factor receptors, and viral envelope proteins. These studies show that heparin-protein interactions depend on the defined patterns and orientations of the sulfo and carboxyl groups along the polysaccharide sequence in the polymer. A correct pattern of basic amino acids in the heparin-binding domain of the proteins is also necessary to ensure the appropriate affinity and specificity of the complex.

Electrostatic interactions play a major role in the binding of heparin to proteins, and basic amino acids such as arginine and lysine are present in the heparin-binding sites of most proteins. A number of studies have been undertaken to determine whether there is a consensus sequence of basic amino acids arranged in a specific way in heparin-binding sites. Comparison of the heparin-binding site amino acid sequence of four proteins: apolipoprotein B, apolipoprotein E, vitronectin, and platelet factor 4 found that these regions are characterized by two consensus sequences of amino acids: XBBXBX and XBBBXXBX, where B is a basic residue and X is a hydropathic residue.

Molecular modeling studies showed that the sequence XBBXBX modeled in a β-strand conformation orients the basic amino acids on one face of the β-strand and the hydropathic residues pointing back into the protein core. Similarly, if the sequence XBBBXXBX is folded into an α-helix, the basic amino acids would be displayed on one side of the helix. Some heparin-binding proteins include the consensus sequences but many others do not contain any of them. This observation suggests that linear patterns of amino acids among the sequences involved in heparin binding may not be necessarily required. Instead, proteins could use a similar spatial structural motif to bind heparin efficiently, in which the basic residues are close in space but not necessarily close in amino acid sequence. Synthetic peptides containing nine contiguous arginine residues bind heparin with affinity similar to that of heparin-binding proteins. Nevertheless, GAG-binding domains made of such extended clusters of basic amino acids rarely occur in natural systems and thus do not appear to present the mechanism chosen in nature to achieve binding.

Heparin-binding sites frequently contain clusters of 1, 2, or 3 basic amino acids (XBnX, where n=1, 2, or 3). Spacing of such clusters with 1 or 2 nonbasic residues (BXmB, where m=1 or 2) is relatively common This finding is in accordance with the observation that heparin-binding proteins usually bind HS in biological systems. Because the charge density of HS is lower, optimal protein binding should involve spaced clusters of basic amino acids. Arginine and lysine are the most frequent residues in heparin- and HS-binding proteins. Although both amino acids have a positive charge at physiological pH, arginine binds heparin ≈2.5× more tightly. Arginine forms more stable hydrogen bonds as well as stronger electrostatic interactions with sulfo groups. Nonbasic residues might also play an important role in heparin-protein interactions. Among them, serine and glycine have been found to be the most frequent nonbasic residues in heparin-binding peptides. Both have small side chains, providing minimal steric constrains and good flexibility for peptide interaction with GAG.

Binding studies and measurement of biological activity of wild-type and mutant forms of heparin binding domains can enable detailed characterization of heparin-binding sites on proteins. Information regarding the structure, affinity, kinetics, and thermodynamics can be obtained through the combination of different structural, biochemical, and genetic techniques. Affinity chromatography is the most commonly used technique in studying the binding affinity of heparin-protein interactions. X-ray crystallography and NMR spectroscopy have also been used particularly in regard to studying structure. Surface plasmon resonance is a powerful technique that has made it possible to study the kinetics of GAG-protein interaction, whereas isothermal titration calorimetry provides thermodynamic data. Using the combination of these and other techniques, a large number of heparin-binding sites have been studied, including growth factors, chemokines, and proteins involved in the anticoagulant system

A heparin-binding domain is a sequence that confers a molecule comprising the heparin-binding domain with the ability to bind heparin. Therefore, the term heparin-binding domain as used herein is non-limiting.

### Angiogenesis

Angiogenesis, which involves the growth or sprouting of new microvessels from pre-existing vasculature, and vasculogenesis, which involves de novo vascular growth, is essential to many physiological and pathological conditions, including embryogenesis, cancer, rheumatoid arthritis, diabetic retinopathy, obesity, atherosclerosis, ischemic heart and limb disease, and wound healing. Over seventy diseases have been identified as angiogenesis dependent (Carmeliet, Nature, 438:932-6, 2005). Under physiological conditions, the growth of new microvessels is tightly regulated and orchestrated by maintaining a balance between endogenous pro- and anti-angiogenic factors. Tipping the balance of this regulation may lead to either excessive neovascularization, as in cancer, age-related macular degeneration, and rheumatoid arthritis, or insufficient vascularization, as in ischemic heart and limb disease, ischemic brain, and neural degeneration.

Angiogenesis is a complex multistep process that involves interactions between endothelial cells (EC), pericytes, vascular smooth muscle cells, and stromal cells (e.g., stem cells and parenchymal cells). These interactions occur through secreted factors, such as vascular endothelial growth factor (VEGF), platelet-derived growth factor (PDGF), basic fibroblast growth factor (bFGF or FGF-2) and angiopoietins, as well as through cell-cell and cell-extracellular matrix (ECM) interactions. Endothelial cell-ECM interactions regulate numerous processes that are critical for angiogenesis, including endothelial cell migration, proliferation, differentiation and apoptosis. Angiogenic processes include network stabilization and remodeling that may involve the recruitment of stromal cells, as well as the pruning of some vessels. In many cases, angiogenesis occurs as a response to hypoxia. A transcription factor called hypoxia-inducible factor, HIF1α, has been demonstrated to act as an oxygen sensor whose activity leads to upregulation of VEGF in parenchymal and stromal cells (Semenza, Physiology (Bethesda), 19:176-82, 2004). VEGF is secreted as a homodimer in the form of several heparin-binding and non-heparin-binding splice-variant isoforms; it diffuses through the interstitial space and can bind to the endothelial cell receptors VEGFR1 and VEGFR2, as well as co-receptors such as Neuropilin-1, thus initiating a signal transduction cascade that leads to endothelial cell proliferation and migration. The production of endothelial cell matrix metalloproteinases, MMPs, increases as a result of endothelial cell activation; MMPs are necessary for selectively clipping the capillary basement membrane and the ECM, which constitute physical barriers to endothelial cell migration and capillary sprouting. MMPs and their associated molecules also play a crucial role in uncovering cryptic sites of the ECM proteins, a number of which have been identified as anti-angiogenic (Davis et al., Anat Rec, 268:252-75, 2002; Folkman, Annu Rev Med, 57:1-18, 2006; Rundhaug, J Cell Mol Med, 9:267-85, 2005; Schenk and Quaranta, Trends Cell Biol, 13:366-75, 2003), and in processing cell-surface receptors (Mott and Werb, Curr Opin Cell Biol, 16:558-64, 2004).

### Angiogenesis Related Diseases

It has been discovered that agents can be loaded into platelets to be incorporated into sites of active angiogenesis or blood vessel injury (i.e. they selectively migrate to such locations). Accordingly, one can target therapeutic agents to such sites by the present invention.

The present invention provides a method for regulating angiogenesis in a patient in need of a change in the rate of angiogenesis at a selected site. The change in angiogenesis necessary may be a reduction or an enhancement of angiogenesis. This is determined by the disorder to be treated. In accordance with the method of the present invention, an effective amount of a platelet loaded with an agent to accomplish the desired result is administered to the patient. To enhance angiogenesis, for example in the treatment of cerebrovascular ischemia, renal ischemia, pulmonary ischemia, limb ischemia, ischemic cardiomyopathy and myocardial ischemia, platelets loaded with one or more types of growth factors are administered.

Where the processes regulating angiogenesis are disrupted, pathology may result. Such pathology affects a wide variety of tissues and organ systems. Diseases characterized by excess or undesirable angiogenesis are susceptible to treatment with therapeutic agents described herein.

Excess angiogenesis in numerous organs is associated with cancer and metastasis, including neoplasia and hematologic malignancies.

Angiogenesis-related diseases and disorders are commonly observed in the eye where they may result in blindness. Such disease include, but are not limited to, age-related macular degeneration, choroidal neovascularization, persistent hyperplastic vitreous syndrome, diabetic retinopathy, and retinopathy of prematurity (ROP).

A number of angiogenesis-related diseases are associated with the blood and lymph vessels including transplant arteriopathy and atherosclerosis, where plaques containing blood and lymph vessels form, vascular malformations, DiGeorge syndrome, hereditary hemorrhagic telangiectasia, cavernous hemangioma, cutaneous hemangioma, and lymphatic malformations.

Other angiogenesis diseases and disorders affect the bones, joints, and/or cartilage include, but are not limited to, arthritis, synovitis, osteomyelitis, osteophyte formation, and HIV-induced bone marrow angiogenesis.

The gastro-intestinal tract is also susceptible to angiogenesis diseases and disorders. These include, but are not limited to, inflammatory bowel disease, ascites, peritoneal adhesions, and liver cirrhosis.

Angiogenesis diseases and disorders affecting the kidney include, but are not limited to, diabetic nephropathy (early stage: enlarged glomerular vascular tufts).

Excess angiogenesis in the reproductive system is associated with endometriosis, uterine bleeding, ovarian cysts, ovarian hyperstimulation.

In the lung, excess angiogenesis is associated with primary pulmonary hypertension, asthma, nasal polyps, rhinitis, chronic airway inflammation, cystic fibrosis.

Diseases and disorders characterized by excessive or undesirable angiogenesis in the skin include psoriasis, warts, allergic dermatitis, scar keloids, pyogenic granulomas, blistering disease, Kaposi's sarcoma in AIDS patients, systemic sclerosis.

Obesity is also associated with excess angiogenesis (e.g., angiogenesis induced by fatty diet). Adipose tissue may be reduced by the administration of angiogenesis inhibitors.

Excess angiogenesis is associated with a variety of auto-immune disorders, such as systemic sclerosis, multiple sclerosis, Sjögren's disease (in part by activation of mast cells and leukocytes). Undesirable angiogenesis is also associated with a number of infectious diseases, including those associated with pathogens that express (lymph)-angiogenic genes, that induce a (lymph)-angiogenic program or that transform endothelial cells. Such infectious disease include those bacterial infections that increase HIF-1 levels, HIV-Tat levels, antimicrobial peptides, levels, or those associated with tissue remodeling.

Infectious diseases, such as viral infections, can cause excessive angiogenesis which is susceptible to treatment with agents of the invention. Examples of viruses that have been found in humans include, but are not limited to, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus;

enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. Ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaviruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

The present invention provides methods of treating diseases and/or disorders or symptoms thereof associated with excess or undesired angiogenesis, which comprise administering a therapeutically effective amount of a pharmaceutical composition comprising a platelet of the invention loaded with a therapeutic compound to a subject (e.g., a mammal, such as a human). Thus, one embodiment is a method of treating a subject suffering from or susceptible to an angiogenesis-related disease or disorder or symptom thereof. The method includes the step of administering to the mammal a therapeutic amount of a platelet of the invention loaded with a therapeutic compound sufficient to treat the disease or disorder or symptom thereof (e.g., to prevent or reduce angiogenesis) under conditions such that the disease or disorder is treated.

The methods herein include administering to the subject (including a subject identified as in need of such treatment) an effective amount of a platelet of the invention loaded with one or more therapeutic compounds, or a composition comprising a singularly loaded platelet (having only one type of cargo), composition comprising a mixture loaded platelet (having a variety of types of cargos), or a composition comprising multiple types of loaded platelets described herein to produce such effect. The amount of cargo loaded may be a small amount (i.e., the same amount as normally present or less than that normally present in a platelet) or a large amount (i.e., 2 to 1000 times as much as normally present in a platelet, e.g., up to two times, five times, ten times, twenty-five, fifty times, 100 times, 200 times, 300 times, 400 times, 500 times, 600 times, 700 times, 800 times, 900 times, and 1000 times). An individual platelet may be loaded up to its maximal capacity. The specific composition can vary among doses such that the amount of loaded platelets, the amount of cargo loaded in platelets, and the types of cargo(s) loaded can vary. Consequently, during a first dose, a subject may receive a composition including a single type of loaded platelet which includes a low amount of a first cargo. During a second dose, the subject may receive a composition including two types of loaded platelets with one containing a high amount of the first cargo and a low amount of a second cargo. During a third dose, the composition may include the same composition as the second dose. And so forth. Thus, the combination of possible compositions and dosing regimens is vast. See, e.g., FIG. 21.

It is noteworthy, that by loading a platelet with a large amount of a cargo(s), a composition is capable of providing localized delivery of a therapeutic agent at a higher local concentration than by other known methods.

A dosing regimen may approximate a naturally occurring process (e.g., wound healing as shown in FIG. 9, top). Alternately, it may deviate from the natural process to obtain an un-natural outcome.

Identifying a subject in need of such treatment can be in the judgment of a subject or a health care professional and can be subjective (e.g. opinion) or objective (e.g. measurable by a test or diagnostic method). The therapeutic methods of the invention (which include prophylactic treatment) in general comprise administration of a therapeutically effective amount of platelet of the invention loaded with a therapeutic compound to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The platelets described herein may be also used in the treatment of any other disorders in which angiogenesis may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with angiogenesis, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Treatment of Neoplasia

The methods of the invention are particularly well suited for the treatment of neoplasias. By “neoplasia” is meant a disease that is caused by or results in inappropriately high levels of cell division, inappropriately low levels of apoptosis, or both. For example, cancer is an example of a proliferative disease. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors, such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

### Angiogenesis Assays

The biological activity of therapeutic agents of the invention is characterized using any method for assaying angiogenic activity known in the art. In vitro angiogenesis assays have been described in detail in recent reviews (Akhtar et al., Angiogenesis, 5:75-80, 2002; Auerbach et al., Cancer Metastasis Rev, 19:167-72, 2000; Auerbach et al., Clin Chem, 49:32-40, 2003; Staton et al., Int J Exp Pathol, 85:233-48, 2004; Vailhe et al., Lab Invest, 81:439-52, 2001). There are a number of different endothelial cell lineages that have been used in angiogenesis assays: bovine aortic, bovine retinal, rat and mouse microvascular, human aortic, human bladder microvascular, human cardiac microvascular, human dermal microvascular, human lung microvascular and human umbilical vein endothelial cells. All of these endothelial cells are capable of differentiating in vitro and forming capillary-like structures. This process occurs when the cells are cultured in a monolayer of extracellular matrix components, such as the Matrigel™ (extracellular matrix material similar to basement membrane), type I collagen, fibronectin or laminin An endothelial cell lineage that is commonly used for testing the angiogenic response is the human umbilical vein endothelial cells (HUVECs). The National Cancer Institute (NCI) has issued guidelines for testing the anti-angiogenic efficacy of novel agents (at the World Wide Web (www) dtp.nci.nih.gov/aa-resources/aa_index.html); they include proliferation, migration and tube formation assays using HUVECs.

Initially the anti-angiogenic effect of selected standard agents is assessed as a positive control by adding them into the wells containing cultured endothelial cells. Such standard anti-angiogenic agents include the fumigillin analog TNP-470 and paclitaxel that are available by request from NCI. The standard cell culture medium is usually used as a negative control. The experiments described below are repeated several times as required to obtain statistically significant and reproducible results. Once the platform is calibrated and tested for the known agents, the novel inhibitors are tested.

A recent review identified over seventy disease conditions that involve angiogenesis, about half of those characterized by abnormal or excessive angiogenesis or lymphangiogenesis (Carmeliet, Nature, 438:932-6, 2005). Agents identified as having anti-angiogenic activity are optionally tested in in vivo assays using animal models that exhibit abnormal or excessive angiogenesis or lymphangiogenesis.

### Cell Proliferation Assay

In these assays anti-angiogenic agents are tested for their ability to alter endothelial cell proliferation. A reduction in endothelial cell proliferation identifies an agent that inhibits angiogenesis. The viability and metabolic activity of the cells is measured in the presence of the anti-angiogenic peptides at different concentrations and various time steps. In one example, a cell proliferation reagent, MTT, is used in a substrate/assay that measures the metabolic activity of viable cells. The assay is based on the reduction of the yellow tetrazolium salt, MTT, by viable, metabolically active cells to form the insoluble purple formazan crystals, which are solubilized by the addition of a detergent. MTT is a colorimetric, non-radioactive assay that can be performed in a microplate. It is suitable for measuring cell proliferation, cell viability or cytotoxicity. The procedure involves three steps. First, the cells are cultured in a multi-well plate and then incubated with the yellow MTT for approximately two to four hours. During this incubation period, viable cells convert, in their mitochondria, the yellow MTT to the purple formazan crystals. The second step involves the solubilization of the crystals. A detergent solution is added to lyse the cells and solubilize the colored crystals. The final step of the assay involves quantifying changes in proliferation by measuring the changes in the color after lysing the cells. The samples are read using an ELISA plate reader at a wavelength of 570 nm. The amount of color produced is directly proportional to the number of viable cells present in a particular well. Other proliferation assays include WST-1, XTT, Trypan Blue, Alamar Blue and BrdU. In contrast to the MTT assay, in the WST-1 assay the formazan crystals do not need to be solubilized by the addition of a detergent; they are soluble to the cell medium.

In another example, cell proliferation is assayed by quantitating bromodeoxyuridine (BrdU) incorporation into the newly synthesized DNA of replicating cells. The assay is a cellular immunoassay that uses a mouse monoclonal antibody directed against BrdU. The procedure involves four steps. First, the cells are cultured in a microtiterplate and pulse-labeled with BrdU. Only proliferating cells incorporate BrdU into their DNA. The cells are then fixed in a denaturing solution. The genomic DNA is denatured, exposing the incorporated BrdU to immunodetection. The BrdU label is located in the DNA with a peroxidase-conjugated anti-BrdU antibody. The antibody is quantitated using a peroxidase substrate. To test anti-proliferative effects of the selected peptides, the endothelial cells are incubated in the presence of varying amounts of the peptides for different time intervals. After labeling of the cells with BrdU the cell proliferation reagent WST-1 is added, and the cells are reincubated. The formazan product is quantified at 450 nm with an absorbance reader. Subsequently, BrdU incorporation is determined using the colorimetric cell proliferation ELISA, BrdU. The results of this assay indicate the effects of the anti-angiogenic peptides either on DNA synthesis (anti-proliferative) or the metabolic activity (pro-apoptotic) of the cell. Kits for implementing these techniques are commercially available.

Preferably, an agent of the invention reduces cell proliferation by at least about 5%, 10%, 20% or 25%. More preferably, cell proliferation is reduced by at least 50%, 75%, or even by 100%.

### Cell Apoptosis and Cell Cycle Assay

Agents having anti-angiogenic activity can also be identified in assay that measure the effect of a candidate agent on cell proliferation and survival using a mitogenic assay (incorporation of thymidine, or 5-bromodeoxyuridine) that measures alterations in cell number (direct counts or indirect colorimetric evaluation). Agents that reduce cell proliferation, cell survival, or that increase cell death are identified as having anti-angiogenic activity. Cell death by apoptosis can be measured using a TUNEL assay or by analyzing the expression of apoptosis markers, such as the caspases and annexin V (Fennell et al., J Biomol Screen, 11:296-302, 2006; Loo and Rillema, Methods Cell Biol, 57:251-64, 1998; Otsuki et al., Prog Histochem Cytochem, 38:275-339, 2003).

A number of methods have been developed to study apoptosis in cell populations. Apoptosis is a form of cell death that is characterized by cleavage of the genomic DNA into discrete fragments prior to membrane disintegration. Because DNA cleavage is a hallmark for apoptosis, assays that measure prelytic DNA fragmentation are especially attractive for the determination of apoptotic cell death. DNA fragments obtained from cell populations are assayed on agarose gels to identify the presence of absence of “DNA ladders” or bands of 180 bp multiples, which form the rungs of the ladders, or by quantifying the presence of histone complexed DNA fragments by ELISA.

Other indicators of apoptosis include assaying for the presence caspases that are involved in the early stages of apoptosis. The appearance of caspases sets off a cascade of events that disable a multitude of cell functions. Caspase activation can be analyzed in vitro by utilizing an enzymatic assay. Activity of a specific caspase, for instance caspase 3, can be determined in cellular lysates by capturing of the caspase and measuring proteolytic cleavage of a suitable substrate that is sensitive to the specific protease (Fennell et al., J Biomol Screen, 11:296-302, 2006; Loo and Rillema, Methods Cell Biol, 57:251-64, 1998; Otsuki et al., Prog Histochem Cytochem, 38:275-339, 2003). Agents that increase caspase activity or DNA fragmentation in endothelial cells are identified as useful in the methods of the invention.

In addition to in vitro techniques, apoptosis can be measured using flow cytometry. One of the simplest methods is to use propidium iodide (PI) to stain the DNA and look for sub-diploid cells (Fennell et al., J Biomol Screen, 11:296-302, 2006; Loo and Rillema, Methods Cell Biol, 57:251-64, 1998; Otsuki et al., Prog Histochem Cytochem, 38:275-339, 2003).

The most commonly used dye for DNA content/cell cycle analysis is propidium iodide (PI). PI intercalates into the major groove of double-stranded DNA and produces a highly fluorescent adduct that can be excited at 488 nm with a broad emission centered around 600 nm. Since PI can also bind to double-stranded RNA, it is necessary to treat the cells with RNase for optimal DNA resolution. Other flow cytometric-based methods include the TUNEL assay, which measures DNA strand breaks and Annexin V binding, which detects relocation of membrane phosphatidyl serine from the intracellular surface to the extracellular surface.

### Cell Migration and Invasion Assay

Another anti-angiogenic activity is the ability to reduce endothelial cell migration towards an attractant that is present in a chemotactic gradient, such as a growth factor gradient. Endothelial cell motility or migration can be assessed using the Boyden chamber technique (Auerbach et al., Cancer Metastasis Rev, 19:167-72, 2000; Auerbach et al., Clin Chem, 49:32-40, 2003; Taraboletti and Giavazzi, Eur J Cancer, 40:881-9, 2004). In one example, a Boyden chamber assay is used to test endothelial cell migration from one side of the chamber in the presence of an activator. In brief, the lower compartment of the Boyden chamber is separated from the upper (containing the endothelial cells) by a matrix-coated polycarbonate filter with pores small enough to allow only the active passage of the cells (3-8 μm pore size). The matrix may include, for example, extracellular matrix proteins, such as collagen, laminin and fibronectin. Activators include but are not limited to growth factors, such as vascular endothelial growth factor and fibroblast growth factor-2 or conditioned medium (e.g. from tumor cells or NIH-3T3 fibroblasts). Migration typically occurs rapidly typically within four to twenty hours cells have migrated through the filter. The number of migrating cells is quantified using a cell-permeable fluorescent dye in the presence or absence of an inhibitor; it can also be quantified by any means of cell counting. A fluorescence plate reader is used to quantify the migrating cells by measuring the amount of fluorescence and directly correlating it to cell number. A decrease in cell migration identifies a peptide that inhibits angiogenesis. Preferably, cell migration or motility is reduced by at least about 5%, 10%, 20% or 25%. More preferably, cell migration or motility is reduced by at least about 50%, 75%, or even by 100%.

In other embodiments, anti-angiogenic agents of the invention alter the invasiveness of an endothelial cell, for example, by reducing the ability of an endothelial cell to degrade an extracellular matrix component. In one example, an anti-angiogenic inhibitor acts by reducing the proteolytic activity of a matrix metalloproteinase. Methods for assaying protease activity are known in the art. Quantification of the matrix metalloproteinase activity can be accomplished using a zymographic or gelatinase activity assay (Frederiks and Mook, J Histochem Cytochem. 52:711-22, 2004). Preferably, protease activity is reduced by at least about 5%, 10%, 20% or 25%. More preferably, protease activity is reduced by at least about 50%, 75%, or even by 100%.

In another example, the invasive activity of an endothelial cell is measured using a Boyden chamber invasion assay or by measuring phagokinetic tracks. The invasion assay is essentially as described above for the Boyden motility assay, except that the filter is coated with a layer of a matrix several microns thick, usually Matrigel™ or other basement membrane extracts, which the cells must degrade before migrating through the filter (Auerbach et al., Cancer Metastasis Rev, 19:167-72, 2000; Auerbach et al., Clin Chem, 49:32-40, 2003; Taraboletti and Giavazzi, Eur J Cancer, 40:881-9, 2004). Compounds that reduce extracellular matrix degradation or endothelial cell invasiveness are identified as useful in the methods of the invention.

### Tumor Models

Many different in vivo models have been developed to test the activity of potential anti-angiogenic or anti-cancer treatments, specifically on tumor vasculature. Tumors are implanted and can be grown syngeneically; i.e., subcutaneously, orthotopically in a tissue of origin, or as xenografts in immunodeficient mice (Auerbach et al., Clin Chem, 49:32-40, 2003; Staton et al., Int J Exp Pathol, 85:233-48, 2004). Any number of histological analyses may be used to examine the effect of a candidate agent on a blood vessel supplying the tumor. In one embodiment, the blood vessel density of a newly formed vasculature in the tumor is monitored; in another embodiment, the vascular architecture is monitored, for example, by counting the number of vascular branches per vessel unit length. In another embodiment, blood flow through the vasculature is measured.

The tumor models provide a variety of different conditions that can be analyzed to assay the efficacy of a candidate anti-angiogenic agent. For example, the effects of a candidate agent on the stability of a well vascularized vs. a poorly vascularized tumor can be assayed; the effect of a candidate agent on tumors of different origin, for example prostate and breast cancer, renal cell carcinoma, and including those of vascular origin such as the chemically induced hemangiosarcomas and Kaposi's sarcomas, can be analyzed. The study of in vivo tumor models provides the closest approximation of human tumor angiogenesis. Moreover, such models provide the opportunity to study the pharmacokinetics of the candidate drug as well as its efficacy simultaneously in a large scale model and under different administration carriers and strategies.

### Polypeptide Agents

The invention provides methods for optimizing a heparin binding domain amino acid or nucleic acid sequence by producing an alteration in the sequence. Such alterations may include certain mutations, deletions, insertions, or post-translational modifications. The invention further includes analogs of any naturally-occurring polypeptide of the invention. Analogs can differ from a naturally-occurring polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 5, 10, 15 or 20 amino acid residues, preferably at least 25, 50, or 75 amino acid residues, and more preferably more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate (EMS) or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and O-phosphoserine, phosphothreonine. “Amino acid analogs” refer to compounds that have the same basic chemical structure as a naturally occurring amino acid, i.e., a carbon that is bound to a hydrogen, a carboxyl group, an amino group, and an R group, for example, homoserine, norleucine, methionine sulfoxide, methionine methyl sulfonium. Such analogs have modified R groups (for example, norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid. Amino acids and analogs are well known in the art Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” apply to both amino acid and nucleic acid sequences. With respect to particular nucleic acid sequences, “conservatively modified variants” refers to those nucleic acids which encode identical or similar amino acid sequences and include degenerate sequences. For example, the codons GCA, GCC, GCG and GCU all encode alanine. Thus, at every amino acid position where an alanine is specified, any of these codons can be used interchangeably in constructing a corresponding nucleotide sequence. The resulting nucleic acid variants are conservatively modified variants, since they encode the same protein (assuming that is the only alternation in the sequence). One skilled in the art recognizes that each codon in a nucleic acid, except for AUG (sole codon for methionine) and UGG (tryptophan), can be modified conservatively to yield a functionally-identical peptide or protein molecule. As to amino acid sequences, one skilled in the art will recognize that substitutions, deletions, or additions to a polypeptide or protein sequence which alter, add or delete a single amino acid or a small number (typically less than about ten) of amino acids is a “conservatively modified variant” where the alteration results in the substitution of an amino acid with a chemically similar amino acid. Conservative substitutions are well known in the art and include, for example, the changes of alanine to serine; arginine to lysine; asparagine to glutamine or histidine; aspartate to glutamate; cysteine to serine; glutamine to asparagine; glutamate to aspartate; glycine to proline; histidine to asparigine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine. Other conservative and semi-conservative substitutions are known in the art and can be employed in practice of the present invention.

The terms “protein”, “peptide” and “polypeptide” are used herein to describe any chain of amino acids, regardless of length or post-translational modification (for example, glycosylation or phosphorylation). Thus, the terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length, naturally occurring proteins as well as recombinantly or synthetically produced polypeptides that correspond to a full-length naturally occurring protein or to particular domains or portions of a naturally occurring protein. The term also encompasses mature proteins which have an added amino-terminal methionine to facilitate expression in prokaryotic cells.

The polypeptides and peptides of the invention can be chemically synthesized or synthesized by recombinant DNA methods; or, they can be purified from tissues in which they are naturally expressed, according to standard biochemical methods of purification. Also included in the invention are “functional polypeptides,” which possess one or more of the biological functions or activities of a protein or polypeptide of the invention. These functions or activities include the ability to inhibit angiogenesis (e.g., by reducing endothelial cell proliferation, migration, survival, or tube formation). The functional polypeptides may contain a primary amino acid sequence that has been modified from that considered to be the standard sequence of a peptide described herein. Preferably these modifications are conservative amino acid substitutions, as described herein.

In addition to full-length polypeptides, the invention also includes fragments of any one of the polypeptides of the invention. As used herein, the term “a fragment” means at least 5, 10, 13, or 15 amino acids. In other embodiments a fragment is at least 20 contiguous amino acids, at least 21, 22, 23, 24, or 25 contiguous amino acids, or at least 30, 35, 40, or 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein transcription factor/protein transduction domain fusion analogs have a chemical structure designed to mimic the fusion proteins functional activity. Such analogs are administered according to methods of the invention. Fusion protein analogs may exceed the physiological activity of the original fusion polypeptide. Methods of analog design are well known in the art, and synthesis of analogs can be carried out according to such methods by modifying the chemical structures such that the resultant analogs increase the reprogramming or regenerative activity of a reference transcription factor/protein transduction domain fusion polypeptide. These chemical modifications include, but are not limited to, substituting alternative R groups and varying the degree of saturation at specific carbon atoms of a reference fusion polypeptide. Preferably, the fusion protein analogs are relatively resistant to in vivo degradation, resulting in a more prolonged therapeutic effect upon administration. Assays for measuring functional activity include, but are not limited to, those described in the Examples below.

### Recombinant Polypeptide Expression

The invention provides agents coupled to a heparin-binding domain for the loading of platelets. Polypeptides comprising a heparin binding domain are expressed recombinantly, either alone, or as part of a larger fusion protein that includes a therapeutic polypeptide operably linked to a heparin binding domain. If desired, the heparin binding domain can subsequently be cleaved (e.g., enzymatically) from a fusion protein. Where the fusion protein does not interfere with the activity of the polypeptide such cleavage may not be necessary or even desirable. When the therapeutic peptide or fusion protein comprising the peptide contacts an endothelial cell, tissue, or organ comprising such a cell it modulates angiogenesis or has some therapeutic effect. Recombinant polypeptides of the invention are produced using virtually any method known to the skilled artisan. Typically, recombinant polypeptides are produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., Current Protocol in Molecular Biology, New York: John Wiley and Sons, 1997). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (e.g., pET-28) (Novagen, Inc., Madison, Wis). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with Factor Xa.

Alternatively, recombinant polypeptides of the invention are expressed in Pichia Pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is coded for by the AOX1 gene is induced by methanol. The AOX1 promoter can be used for inducible polypeptide expression or the GAP promoter for constitutive expression of a gene of interest.

Once the recombinant polypeptide of the invention is expressed, it is isolated, for example, using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column.

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

### Oligonucleotide Directed Mutagenesis

Oligonucleotide directed mutagenesis can be used in order to modify a single or multiple amino acids that compose the maternal sequence of the predicted anti-angiogenic fragments (Ryu and Nam, Biotechnol Prog, 16:2-16, 2000). Directed mutagenesis is based on the concept that an oligonucleotide encoding a desired mutation is annealed to one strand of a DNA of interest and serves as a primer for initiation of DNA synthesis. In this manner, the mutagenic oligonucleotide is incorporated into the newly synthesized strand. Mutagenic oligonucleotides incorporate at least one base change but can be designed to generate multiple substitutions, insertions or deletions.

Oligonucleotides can also encode a library of mutations by randomizing the base composition at sites during chemical synthesis resulting in degenerate oligonucleotides. The ability to localize and specify mutations is greatly enhanced by the use of synthetic oligonucleotides hybridized to the DNA insert-containing plasmid vector. The general format for site-directed mutagenesis includes several steps. Plasmid DNA containing the template of interest (cDNA) is denatured to produce single-stranded regions. A synthetic mutant oligonucleotide is annealed to the target strand. DNA polymerase is used to synthesize a new complementary strand, and finally DNA ligase is used to seal the resulting nick between the end of the new strand and the oligonucleotide. The resulting heteroduplex is propagated by transformation in E. coli.

### Phage-Displayed Peptide Library Screening

Phage display is one method for in vitro combinatorial biology. The method stems from the observation that peptides fused to certain bacteriophage coat proteins are displayed on the surfaces of phage particles that also contain the cognate DNA (Landon et al., Curr Drug Discov Technol, 1:113-32, 2004).

Phage display describes a selection technique in which a library of variants of an initial peptide (e.g., a peptide described herein), is expressed on the outside of a phage virion, while the genetic material encoding each variant resides on the inside. This creates a physical linkage between each variant protein sequence and the DNA encoding it, which allows rapid partitioning based on binding affinity to a given target molecule by an in vitro selection process called panning In its simplest form, panning is carried out by incubating a library of phage-displayed peptides with a plate containing a culture of cells, such as endothelial cells, washing away the unbound phage, and eluting the specifically bound phage. The eluted phage is then amplified and taken through additional binding/amplification cycles to enrich the pool in favor of specific phenotypes, such as suppression of proliferation, of the cells that are cultured. After three to four rounds, individual clones are characterized by DNA sequencing and ELISA.

Libraries of “fusion phages” are rapidly sorted to obtain clones with desired properties and phages can be readily amplified by passage through a bacterial host. Phage display was first demonstrated with the Escherichia-coli-specific M13 bacteriophage and this remains the most popular platform. Several other E. coli phages have also been adapted for phage display and eukaryotic systems have also been developed.

### Test Compounds and Extracts

In general, agents or peptides are identified from large libraries of natural product or synthetic (or semi-synthetic) extracts or chemical libraries or from polypeptide or nucleic acid libraries, according to methods known in the art. Such candidate polypeptides or the nucleic acid molecules encoding them may be modified to enhance biodistribution, protease resistance, or specificity. The modified peptides are then screened for a desired activity (e.g., angiogenesis modulating activity). Those skilled in the field of drug discovery and development will understand that the precise source of test extracts or compounds is not critical to the screening procedure(s) of the invention. Agents used in screens may include known compounds (for example, known polypeptide therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have angiogenesis modulating activity further fractionation of the positive lead extract is necessary to isolate molecular constituents responsible for the observed effect. Thus, the goal of the extraction, fractionation, and purification process is the careful characterization and identification of a chemical entity within the crude extract that alters angiogenesis (increases or decreases). Methods of fractionation and purification of such heterogeneous extracts are known in the art. If desired, compounds shown to be useful as therapeutics are chemically modified according to methods known in the art.

Alternatively, or in addition, candidate agents may be identified that specifically bind to and inhibit a peptide of the invention. The efficacy of such a candidate compound is dependent upon its ability to interact with the peptide. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate angiogenesis may be assayed by any standard assays (e.g., those described herein).

Potential antagonists include organic molecules, peptides, peptide mimetics, polypeptides, nucleic acid ligands, aptamers, and antibodies that bind to a peptide of the invention and thereby inhibit or extinguish its activity. Potential antagonists also include small molecules that bind to and occupy the binding site of the polypeptide thereby preventing binding to cellular binding molecules, such that normal biological activity is prevented.

In one particular example, a candidate compound that binds to a pathogenicity polypeptide may be identified using a chromatography-based technique. For example, a recombinant polypeptide of the invention may be purified by standard techniques from cells engineered to express the polypeptide, or may be chemically synthesized, once purified the peptide is immobilized on a column. A solution of candidate compounds is then passed through the column, and a compound specific for the peptide is identified on the basis of its ability to bind to the peptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to modulate angiogenesis (e.g., as described herein). Compounds isolated by this approach may also be used, for example, as therapeutics to treat or prevent the onset of a disease or disorder characterized by excess or undesirable angiogenesis. Compounds that are identified as binding to peptides with an affinity constant less than or equal to 1 nM, 5 nM, 10 nM, 100 nM, 1 mM or 10 mM are considered particularly useful in the invention.

High-throughput low-cost screening methods of the invention are useful for identifying polypeptides, biologically active fragments or analogs thereof that can be used to modulate angiogenesis. One skilled in the art appreciates that the effects of a candidate peptide on a cell (e.g., an endothelial cell) are typically compared to a corresponding control cell not contacted with the candidate peptide. Thus, the screening methods include comparing the expression profile, phenotype, or biological activity of a cell modulated by a candidate peptide to a reference value of an untreated control cell.

In one example, candidate peptides are added at varying concentrations to the culture medium of an endothelial cell. The survival, tube formation, apoptosis, proliferation, migration of the cell are assayed as indicators of angiogenesis. Peptides that reduce the survival, tube formation, proliferation, or migration of an endothelial cell are identified as useful anti-angiogenic agents. Alternatively, peptides that enhance the survival, tube formation, proliferation, or migration of an endothelial cell are identified as useful angiogenic agents. In another embodiment, the expression of a nucleic acid molecule or polypeptide characteristic of the vasculature is monitored. Typical cell surface markers include the fibronectin extra-domain B, large tenascin-C isoforms, various integrins, VEGF receptors, prostate specific membrane antigen, endoglin and CD44 isoforms and tumor endothelium marker (TEM). Peptides or other agents that alter the expression of such markers are identified as useful modulators of angiogenesis. An agent that reduces the expression of a characteristic polypeptide expressed in the vasculature is considered useful in the invention; such an agent may be used, for example, as a therapeutic to prevent, delay, ameliorate, stabilize, or treat an injury, disease or disorder characterized by an undesirable increase in neovascularization. In other embodiments, agents that increase the expression or activity of a marker characteristically expressed in an endothelial cell are used to prevent, delay, ameliorate, stabilize, or treat an injury, disease or disorder characterized by a reduction in angiogenesis. Agents identified according to the methods described herein may be administered to a patient in need of angiogenesis modulation. Where such agents are peptides, such as those described herein, one skilled in the art appreciates that the invention further provides nucleic acid sequences encoding these peptides.

An agent useful herein includes any isoform of said agent.

### Therapeutic Methods

Therapeutic agents, polypeptides, peptides, or analogs or fragments thereof, as well as the nucleic acid molecules encoding such molecules are useful for preventing or ameliorating a disease or injury associated with an undesirable increase or decrease in angiogenesis. Diseases and disorders characterized by excess angiogenesis may be treated using the methods and compositions of the invention. Such diseases and disorders include, but are not limited to, neoplasia, hematologic malignancies, rheumatoid arthritis, diabetic retinopathy, age-related macular degeneration, atherosclerosis, and pathologic obesity. In one embodiment, a angiogenesis regulating factor is delivered to one or more endothelial cells at a site of angiogenesis-associated disease or injury.

In other embodiments, a nucleic acid molecule encoding a therapeutic polypeptide of the invention is administered to a cell, tissue, or organ in need of a reduction in angiogenesis. If desired, the peptide is expressed as a fusion with a longer polypeptide. The peptide may then be cleaved from the polypeptide to achieve its desired therapeutic effect. Such cleavage is not required where the fusion protein does not interfere with the polypeptide's biological activity.

### Pharmaceutical Therapeutics

A chemical entity discovered to have medicinal value is useful as a drug or as information for structural modification of existing compounds, e.g., by rational drug design. Such methods are useful for screening compounds having an effect on a variety of conditions characterized by undesired angiogenesis.

For therapeutic uses, the compositions or agents are loaded into platelets, and these compositions may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic agent described herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the disease or disorder. Generally, amounts will be in the range of those used for other agents used in the treatment of other diseases associated with alterations in angiogenesis, although in certain instances lower amounts will be needed because of the increased specificity of the compound. A compound is administered at a dosage that controls the clinical or physiological symptoms associated with angiogenesis as determined by a diagnostic method known to one skilled in the art.

It would be advantageous to administer therapeutic peptides in a formulation that would slow their elimination from the circulation through renal filtration, enzymatic degradation, uptake by the reticulo-endothelial system (RES), and accumulation in non-targeted organs and tissues. In addition, methods for administering agents that limits their widespread distribution in non-targeted organs and tissues allows lower concentrations of the agent to be administered reducing adverse side-effects and providing economic benefits. A variety of methods are available to slow the elimination of agents of the invention. In one embodiment, an implantable device is used to provide for the controlled release of an agent described herein. Such devices are known in the art and include, but are not limited to, polymeric gels and micro-fabricated chips. Some of these devices are already used in the clinic or are being tested in clinical trials (Moses et al., Cancer Cell, 4:337-41, 2003). Various delivery methods for anti-angiogenic agents are tissue specific, e.g., intraocular and periocular injection or gene transfer in the eye (Akiyama et al., J Cell Physiol, 2006; Saishin et al., Hum Gene Ther, 16:473-8, 2005). Numerous reviews on the subject of anti-angiogenic drug delivery are available.

### Formulation of Pharmaceutical Compositions

The administration of a compound for the treatment of treatment of a disease or disorder associated with altered levels of angiogenesis may be by any suitable means that results in a concentration of the therapeutic that, combined with other components, is effective in ameliorating, reducing, or stabilizing a disease or disorder associated with altered levels of angiogenesis (e.g., an amount sufficient to reduce neovascularization). The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that allow for convenient dosing for metronomic therapy that would require taking small doses of the drug several times a week; (vii) formulations that target a disease or disorder associated with altered levels of angiogenesis by using carriers or chemical derivatives to deliver the therapeutic agent to a particular cell type (e.g., endothelial cell) whose function is perturbed in a disease or disorder associated with altered levels of angiogenesis. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active therapeutic(s), the composition may include suitable parenterally acceptable carriers and/or excipients. The active therapeutic(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active angiogenic modulating therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam- nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

VEGFs and their Receptors

VEGFs represent a class of proteins that promote angiogenesis, increase vascular permeability and contribute to endothelial-cell survival in blood and lymphatic vessels. The contribution of VEGFA to cancer progression has been highlighted by the recent approval of the humanized anti-VEGF monoclonal antibody bevacizumab (Avastin®; Genentech) for first-line cancer treatment. The overexpression of VEGFs and VEGF receptors in tumors is well documented. The selective tumor localization of monoclonal antibodies to VEGFA, VEGF receptor 2 and the VEGFA-VEGF receptor 2 complex can be used as an excellent selectivity mechanism for targeting the angiogenic vasculature.

### Combination Therapies

Optionally, an angiogenic modulating therapeutic as described herein may be administered in combination with any other standard active angiogenic modulating therapeutics; such methods are known to the skilled artisan and described in Remington's Pharmaceutical Sciences by E. W. Martin. For example, a platelet of the invention loaded with an anti-angiogenic agent may be administered in combination with any other known anti-angiogenic agent (Folkman, Annu Rev Med. 57:1-18, 2006).

For the treatment of a neoplasia, a platelet of the invention (e.g., loaded with a cytotoxic agent or angiogenesis inhibitor) is administered in combination with any conventional treatment (e.g., chemotherapy, radiotherapy, hormonal therapy, surgery, cryosurgery). A pharmaceutical composition of the invention may, if desired, include one or more chemotherapeutics typically used in the treatment of a neoplasm, such as abiraterone acetate, altretamine, anhydrovinblastine, auristatin, bexarotene, bicalutamide, BMS184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine (BCNU),cisplatin, cryptophycin, cyclophosphamide, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermin, taxol, thalidomide, tretinoin, vinblastine, vincristine, vindesine sulfate, and vinflunine. Other examples of chemotherapeutic agents can be found in Cancer Principles and Practice of Oncology by V. T. Devita and S. Hellman (editors), 6th edition (Feb. 15, 2001), Lippincott Williams & Wilkins Publishers.

### Kits

The invention provides kits for the preparing an isolated platelet of the present invention and for using an isolated platelet in the treatment or prevention of diseases or disorders involving sites of injury, inflammation, or angiogenesis. In one embodiment, the kit includes a therapeutic or prophylactic composition containing an effective amount of platelets loaded with a therapeutic agent in unit dosage form. In some embodiments, the kit comprises a sterile container that contains a therapeutic or prophylactic vaccine; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

A platelet of the invention may be provided with instructions for administering it to a subject having or at risk of developing a disease or condition involving regulation of angiogenesis. The instructions may include information about the use of the composition for the treatment or prevention of the disease or for drug delivery to a tissue in need thereof. In other embodiments, the instructions include at least one of the following: description of the therapeutic agent; dosage schedule and administration for treatment or prevention of the disease or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container. Instructions for preparing an isolated platelet may be included in a kit.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1

### Proteins with Heparin Binding Domains are Actively and Selectively Taken up into Platelets in a Receptor-Independent Process

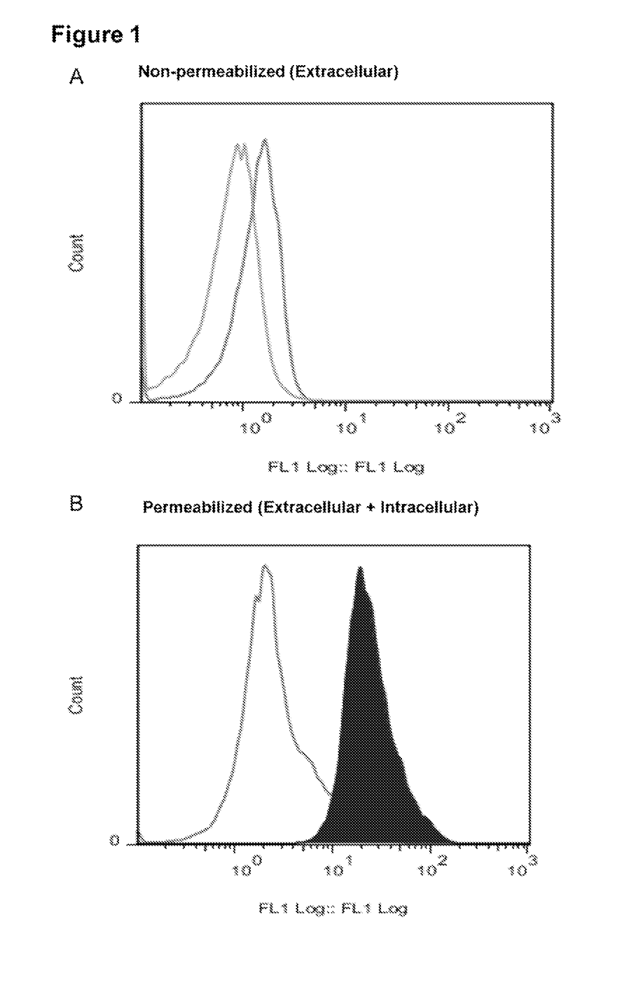

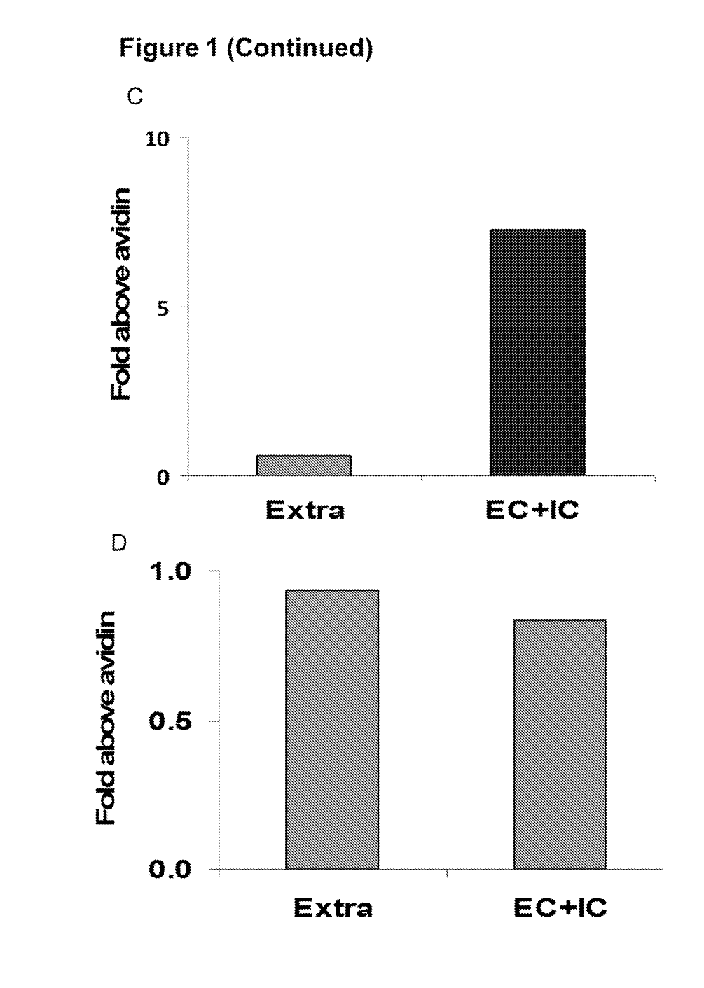

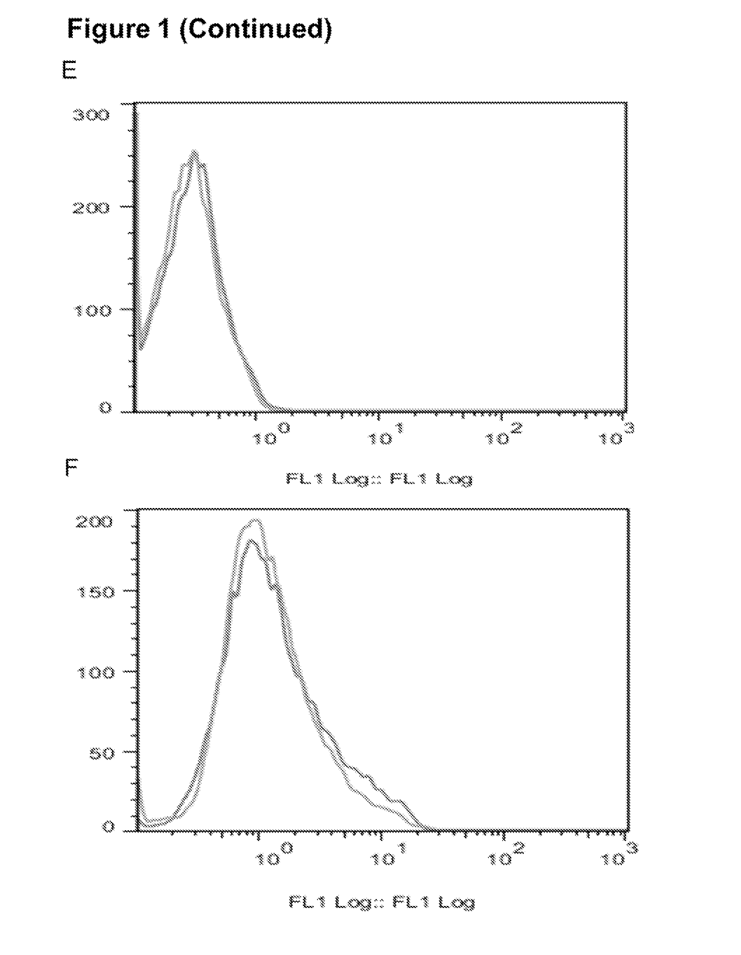

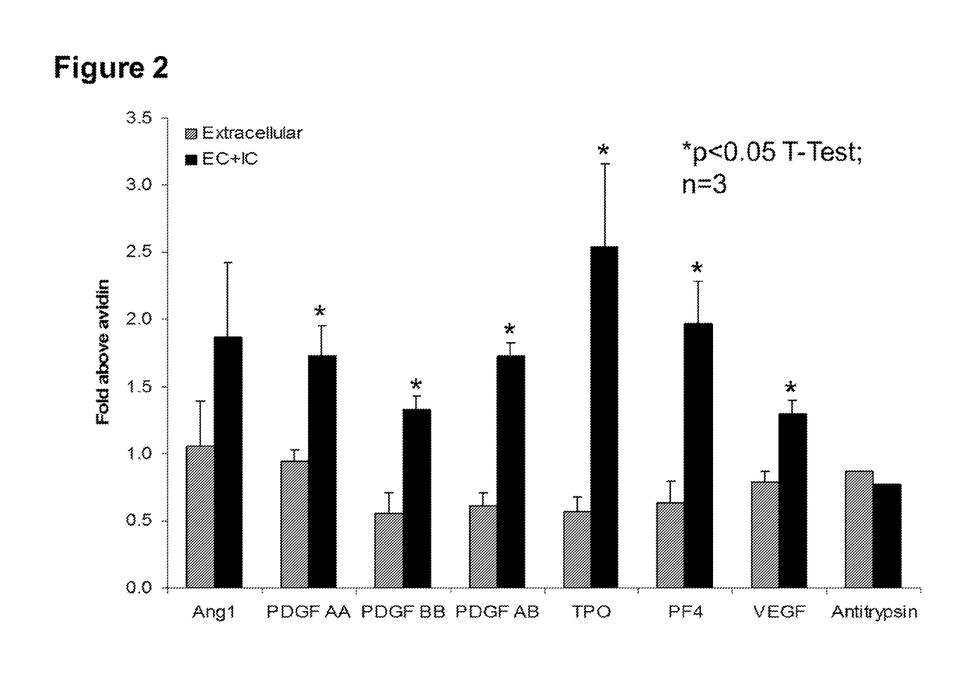

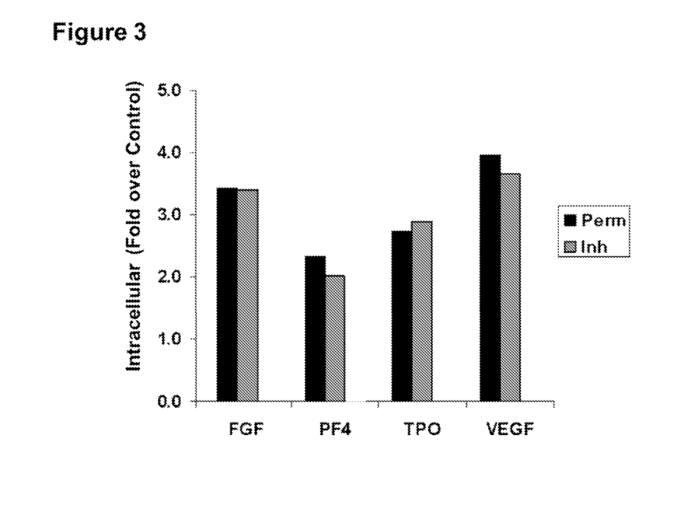

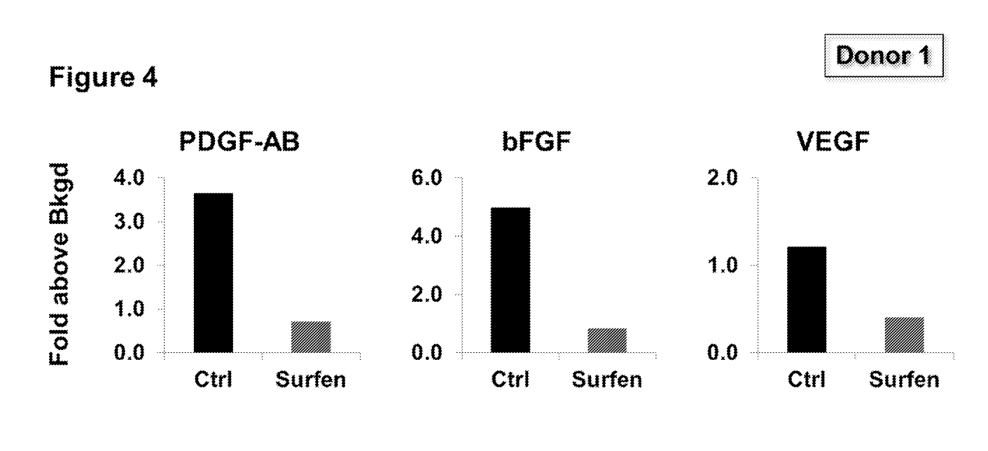

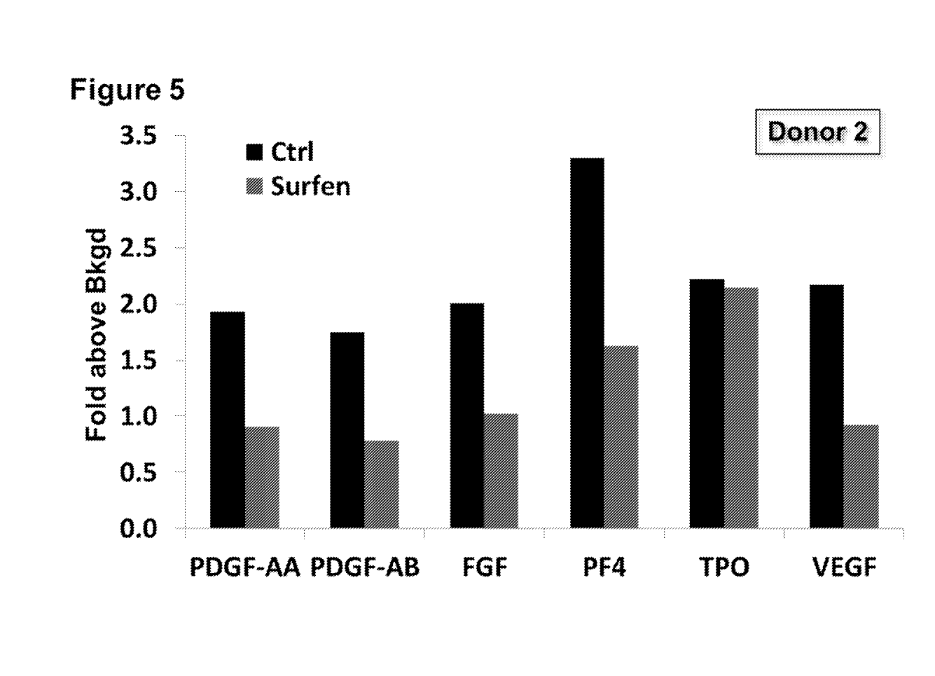

It is known that angiogenesis regulators are sequestered in platelets actively and selectively against the concentration gradient. In in vitro studies, human resting platelets selectively took up PDGF compared to a control (soybean trypsin inhibitor) (FIG. 1). Indeed, the present inventors have loaded 600 times the physiological levels of recombinant bFGF or recombinant PGDF into platelets (data not show); however, this load level does not appear to be the upper limit of a platelet's capacity. As shown by FACS and ICC assays, PDGF is released after permeabilization of the treated cells whereas soybean trypsin inhibitor is not. Various growth factors and chemokines were internalized, including Ang1, PDGF AA, PDGF BB, PDGF AB, TPO, PF4, and VEGF, as shown by FACS analysis (FIG. 2). To study the process of protein uptake into the platelets, individual receptors were inhibited. However, exemplary growth factors and chemokines, including FGF, PF4, TPO, and VEGF, were sequestered in the platelets despite blockade of their receptors (FIG. 3). It was observed that pre-treatment of platelets with Surfen, an inhibitor of heparan sulfate binding, prevented platelet sequestration of angiogenesis regulators, PDGF-AB, bFGF, and VEGF (FIG. 4). However, when thrombopoietin (TPO) was tested in this assay, surfen pre-treatment did not block uptake of TPO (FIG. 5). In this regard, it was appreciated that unlike the other growth factors and chemokines tested TPO was the only one without known heparin binding activity. Without being bound to a particular theory, the results indicated that heparin binding activity of the proteins, which have been characterized as having heparin binding domains, was important for platelet uptake.

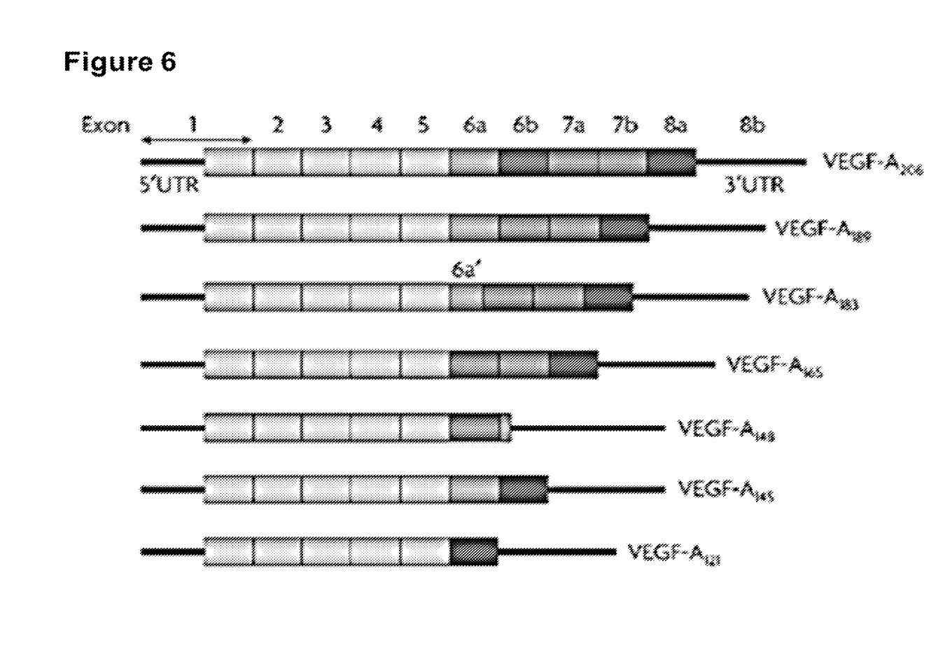

Indeed, this could account for the differential activities, functions, and localization of VEGF isoforms created by alternative splicing of the gene (FIG. 6). VEGF₁₂₁ has a weakly acid character and behaves as a soluble protein that fails to bind heparin presumably because it lacks a heparin binding domain. The predominant molecular species in humans, VEGF₁₆₅, is also diffusible, although an important fraction remains bound to the extracellular matrix, presumably via its heparin binding domain. The VEGF₁₈₉ and the VEGF₂₀₆ are more basic and bind heparin with greater affinity, and they are sequestered in the extracellular matrix. Transcripts encoding VEGF₁₂₁ and VEGF₁₈₉ are detected in the majority of cells and tissues expressing VEGF. In contrast, VEGF₂₀₆ production has only been identified in human and murine fetal liver cDNA libraries.

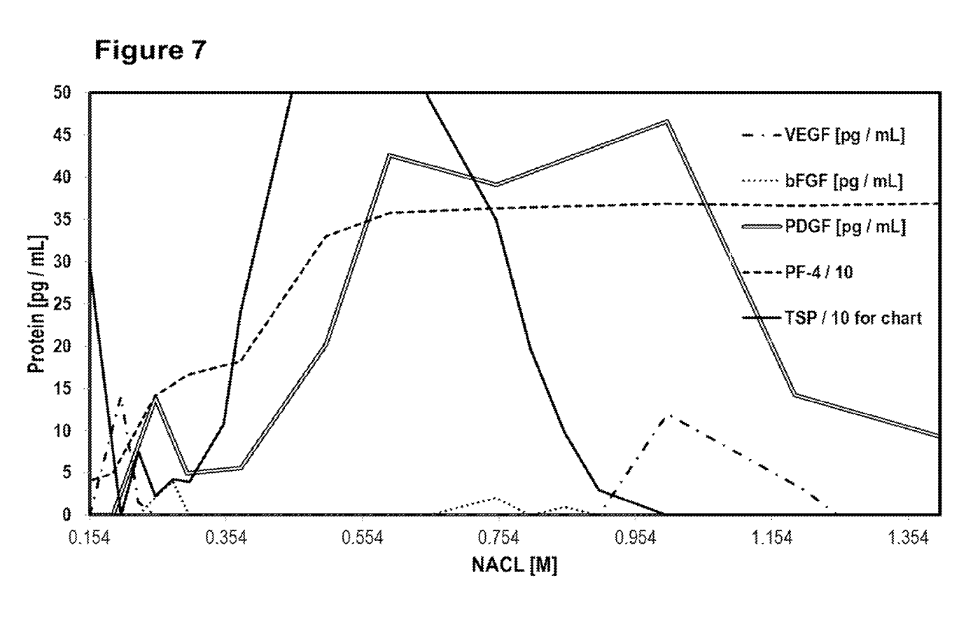

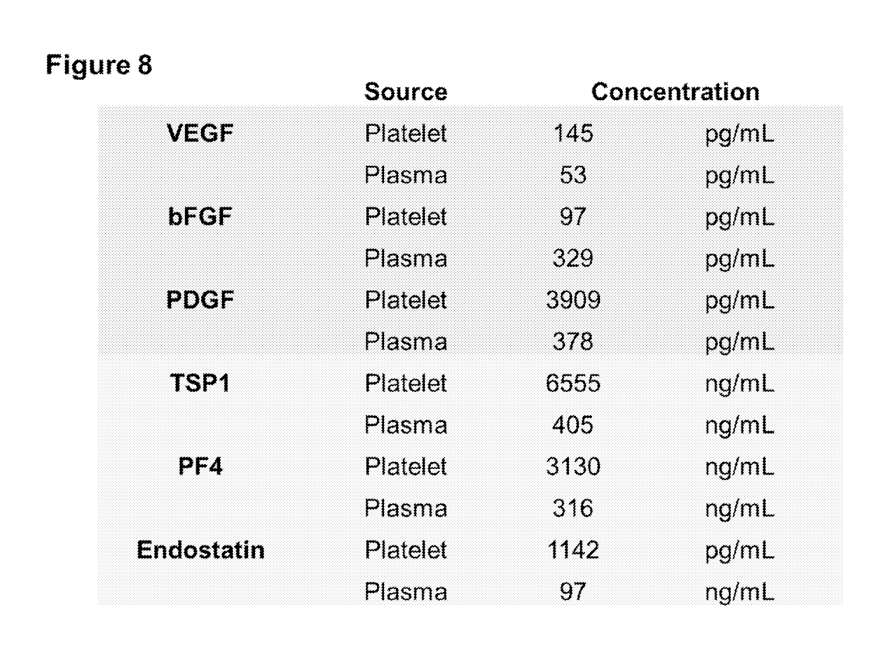

Without being bound to a particular theory, it was hypothesized that differential binding could be a mechanism for regulation of angiogenic regulating factors by platelets. As shown by elution of angiogenesis regulators obtained from a platelet clot from a heparin column, the angiogenesis regulators have different affinities for heparin which corresponds with the timing of their activity (FIG. 7). Despite the concentration of angiogenesis inhibitors in platelets surpassing many fold the concentration of stimulators (FIG. 8), regulation of their release could account for their roles in angiogenic processes in different microenvironments including wound healing and tumor growth (FIG. 9).

### Example 2

### Platelets Loaded with VEGF₁₆₄ or VEGF₁₆₅ Selectively Deliver VEGF to Endothelial (HMVEC) Cells Whereas Platelets Loaded with VEGF₁₂₁ do not Deliver VEGF to MHVEC Cells

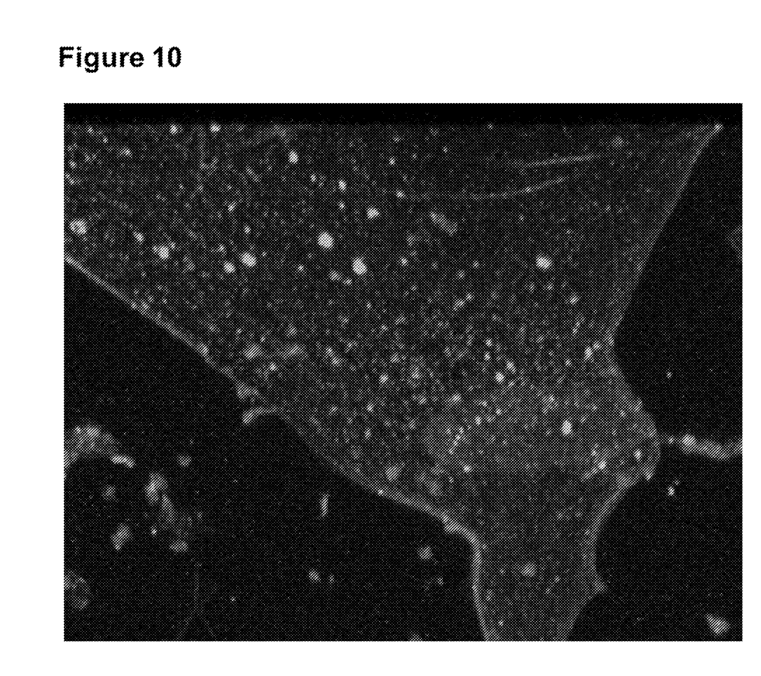

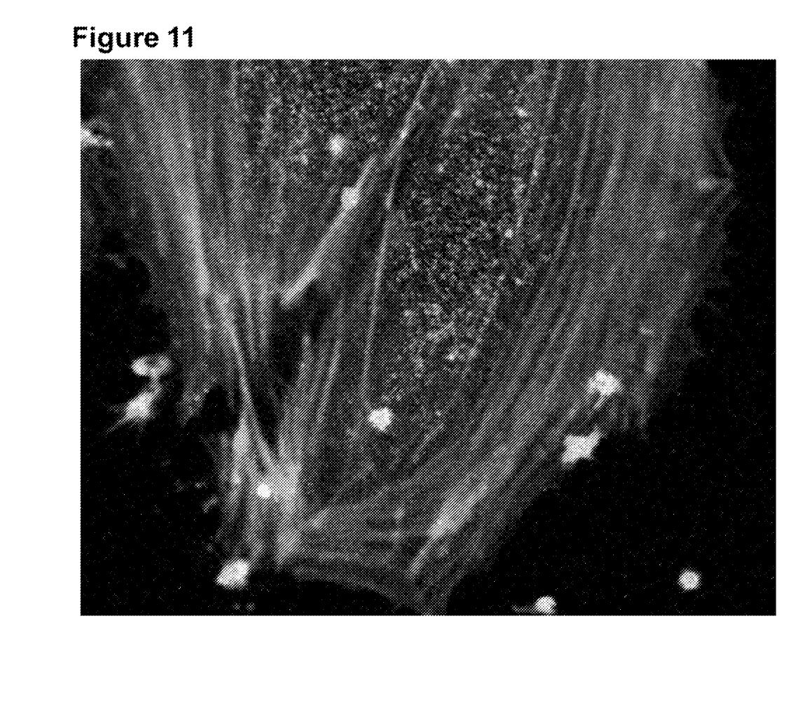

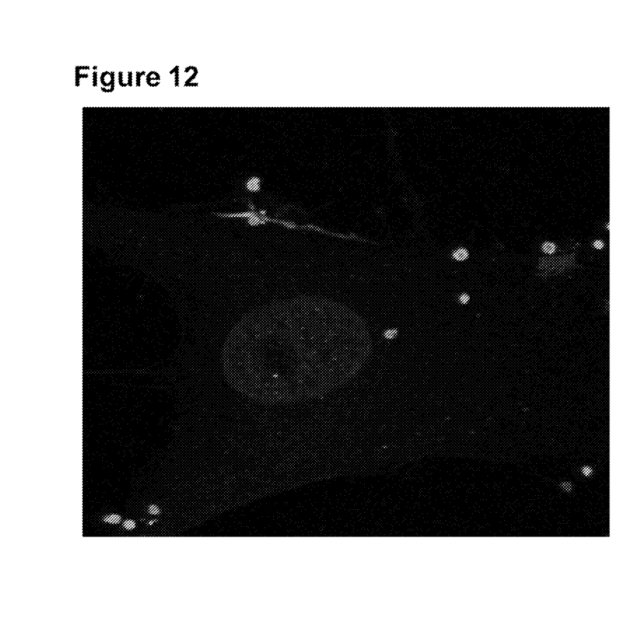

Cultured HMVEC cells were incubated with platelets loaded specifically with VEGF₁₆₅ or loaded non-specifically with VEGF₁₂₁. HMVEC cells incubated with platelets loaded with VEGF₁₆₅ (a human VEGF isoform which comprises a heparin binding domain) had strong internal staining of VEGF; this is consistent with VEGF delivery to HMVEC cells by the loaded platelets. (FIG. 10) Similarly, HMVEC cells incubated with platelets loaded with VEGF₁₆₄ (a murine VEGF isoform which comprises a heparin binding domain) had strong internal staining of VEGF; this is consistent with VEGF delivery to HMVEC cells by the loaded platelets. (FIG. 11) In contrast, HMVEC cells incubated with platelets loaded with VEGF₁₂₁ (which lacks a heparin binding domain) did not have internal staining of VEGF₁₂₁; this is consistent with failure of VEGF delivery to HMVEC cells by the loaded platelets. (FIG. 12)

These data indicate that platelets loaded with an active agent comprising a heparin binding domain can recognize and target a cell and specifically deliver its cargo into the cell. This process occurs via interactions between a loaded platelet and its target cell and is likely to be independent of the cell's context, i.e., whether the cell is in vitro or in vivo.

It is expected that delivery of any active agent to an endothelial cell, depending on the cargo, would be effective in inhibiting certain relevant pathogenic conditions and/or would be effective in promoting recovery.

### Example 3

### Platelets have a Differential Effect on Proliferation of Neuroblastoma (SK-N-SH) and Endothelial (HMVEC) Cells

To understand how platelets interact with the tumor microenvironment, the effect of platelets on proliferation rates of neuroblastoma (SK-N-SH) and endothelial (HMVEC) cells was studied using CyQuant® Proliferation assays (Invitrogen), which measures total DNA content. In black 96 well fluorescent plates (Corning), 3000 cells/well of HMVEC or SK-N-SH were plated and allowed to adhere overnight. The following day, cells received fresh media, and were treated with 30 μl of Platelet Rich Plasma (PRP). Samples were run in octuplet and the experiment was repeated three times.

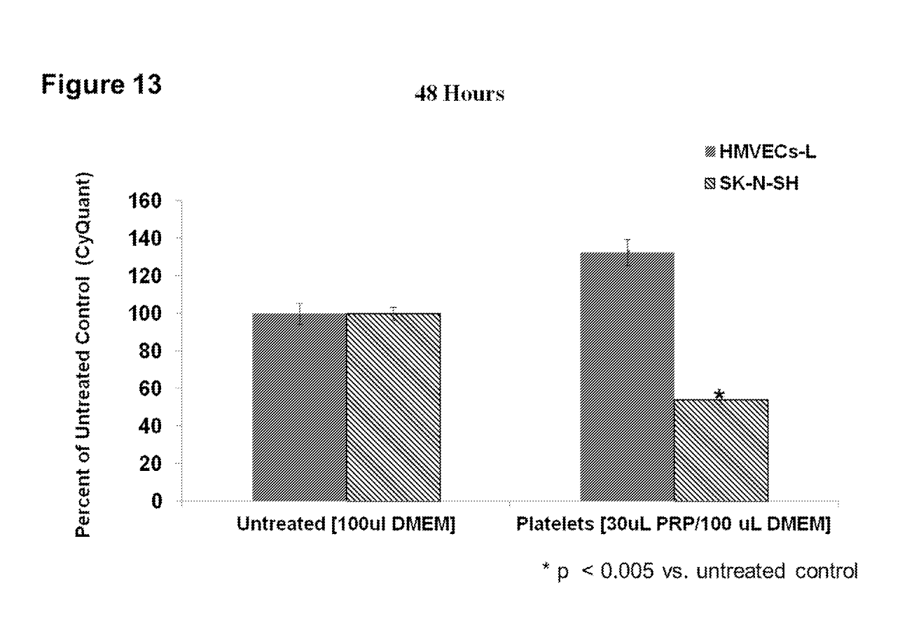

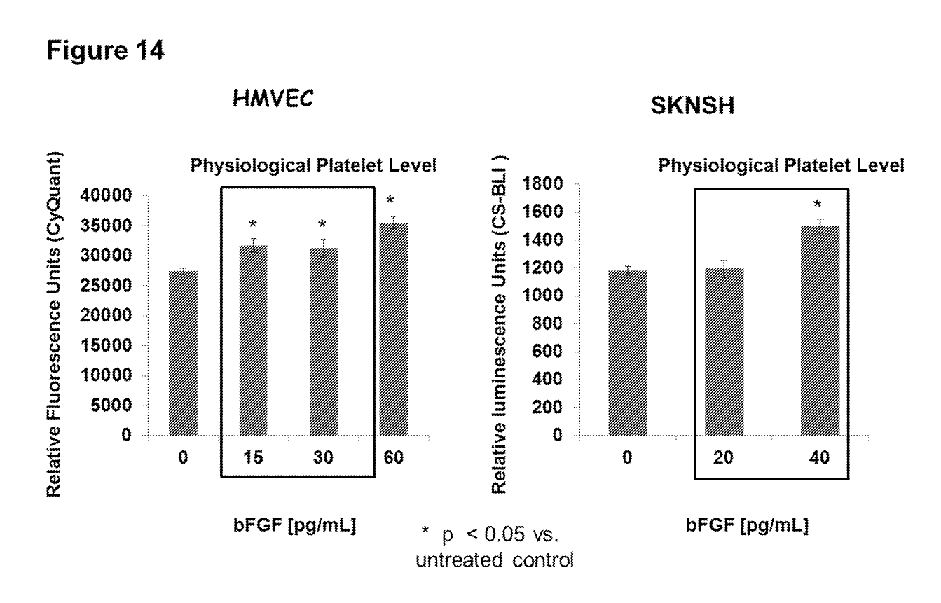

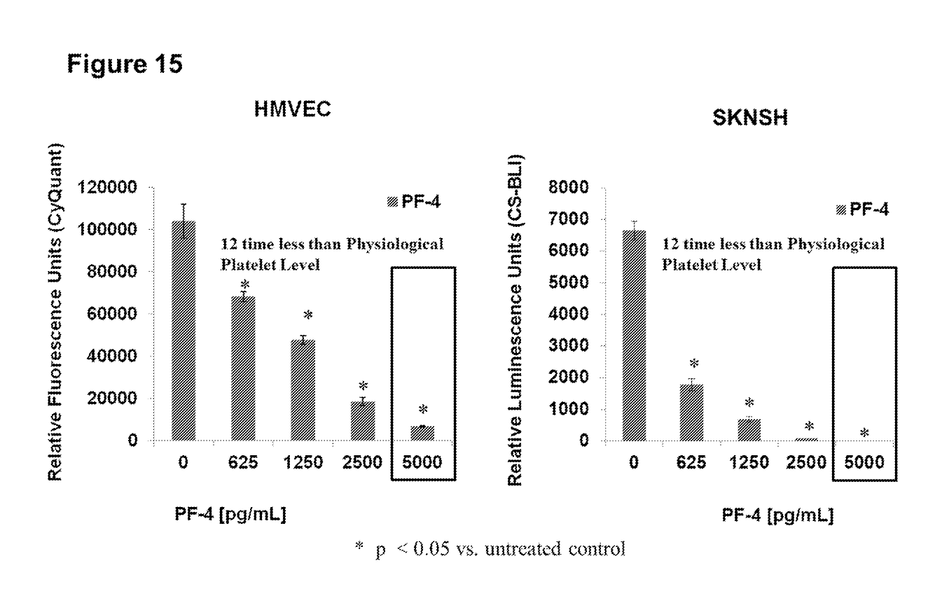

Platelets had a differential effect on the proliferation of neuroblastoma or endothelial cells (FIG. 13). Platelets increased the proliferation of HMVEC-L by 33% when compared to untreated controls. Yet when incubated with SK-N-SH, there was a 47% decrease in proliferation after 48 hours. Serum effects on proliferation were a 6% increase on HMVEC and a 65% decrease on SK-N-SH cells (P<0.005). In contrast, administering individual proteins (e.g., bFGF) did not account for differentially regulated growth in HMVEC and SK-N-SH cells (FIG. 14). Likewise, individual inhibitors had the same effects on both HMVEC and SK-N-SH cells (FIG. 15). Thus, the results indicated the reduced proliferative effect on SK-N-SH neuroblastoma cells was due to treatment with platelets.

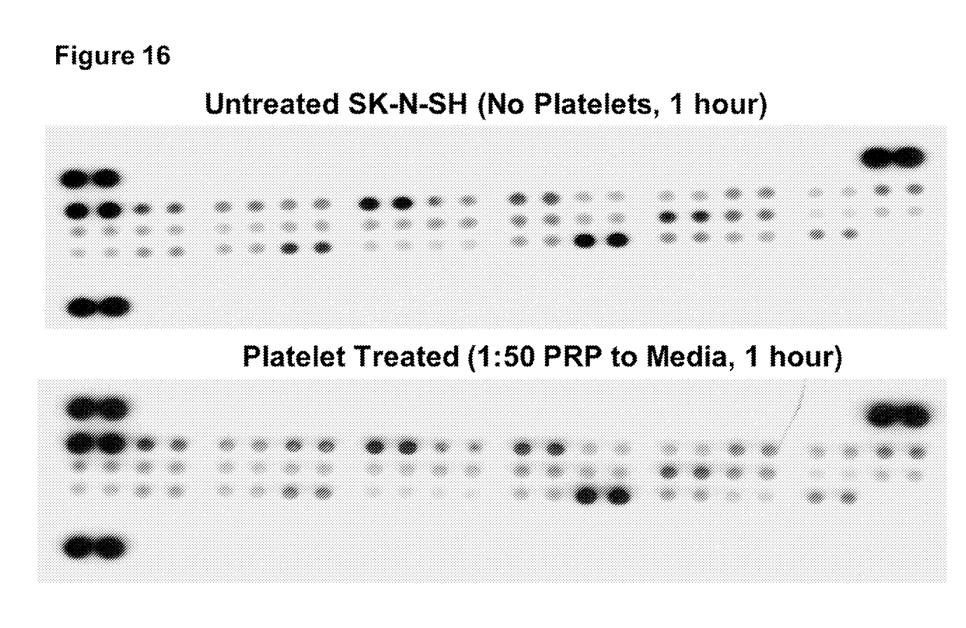

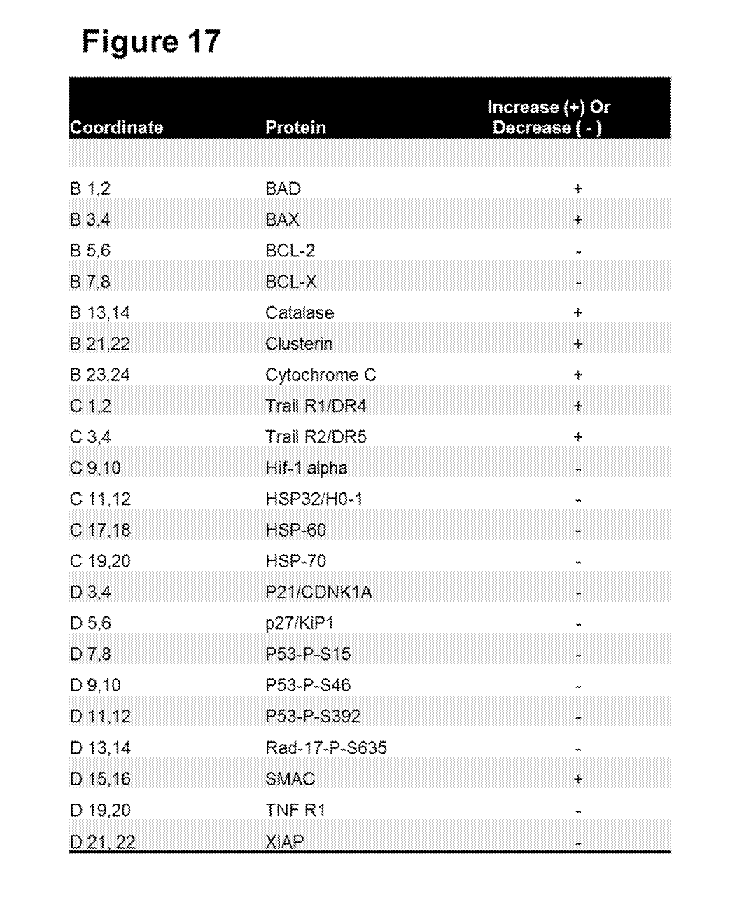

To determine whether cell death was increased in the SK-N-SH neuroblastoma cells, untreated and platelet treated cells were assayed for apoptotic cell markers using an apoptosis array from R and D systems (ARY009). Cells were grown in 10 CM dishes to a confluence of 60%. They were then treated with platelets and collected at 20 min and 60 min time points. The plates were washed with PBS and then cells were scrapped into 1 mL of PBS and put on ice. Pellets were obtained by spinning the cells at 2000 g for 4 min at 4° C. Once cell pellets were obtained, cells were lysed and the apoptosis array protocol was followed according to the manufacturer's specifications. Pierce® ECL reagent was used with Kodak film and were developed by AGFA CP1000 film developer. Films were scanned and arrays were pasted into excel. Images were also loaded into GIMP image software and subtracted from each other to determine which proteins were increased or decreased with platelet treatment. Quantitation of the expression of apoptotic markers indicated that platelets treated with SK-N-SH showed increased apoptosis when compared to untreated controls (FIGS. 16 and 17).

### Example 4

### Platelets have Inhibitory Effects on Other Tumor Cells Types

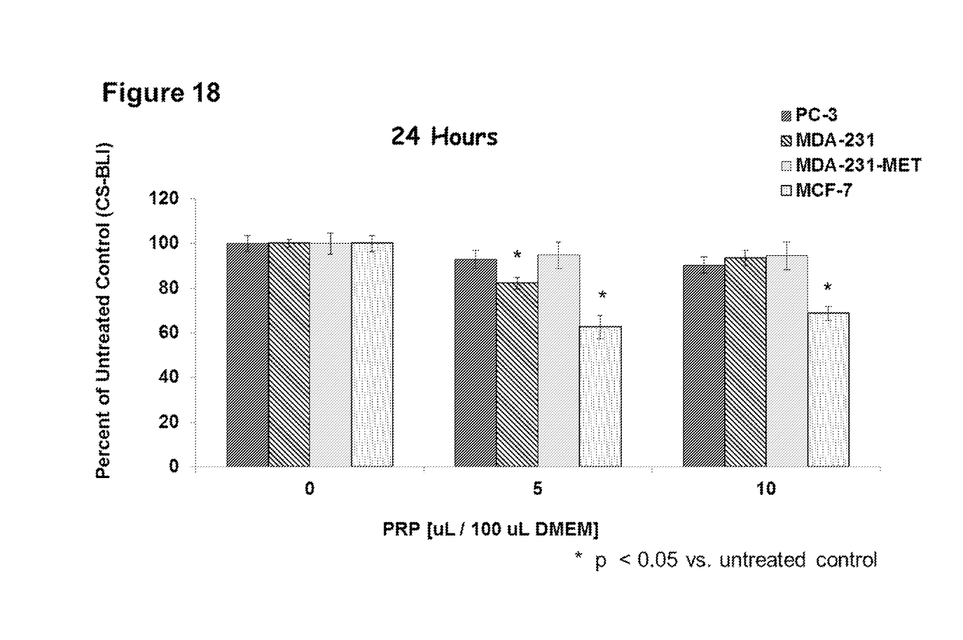

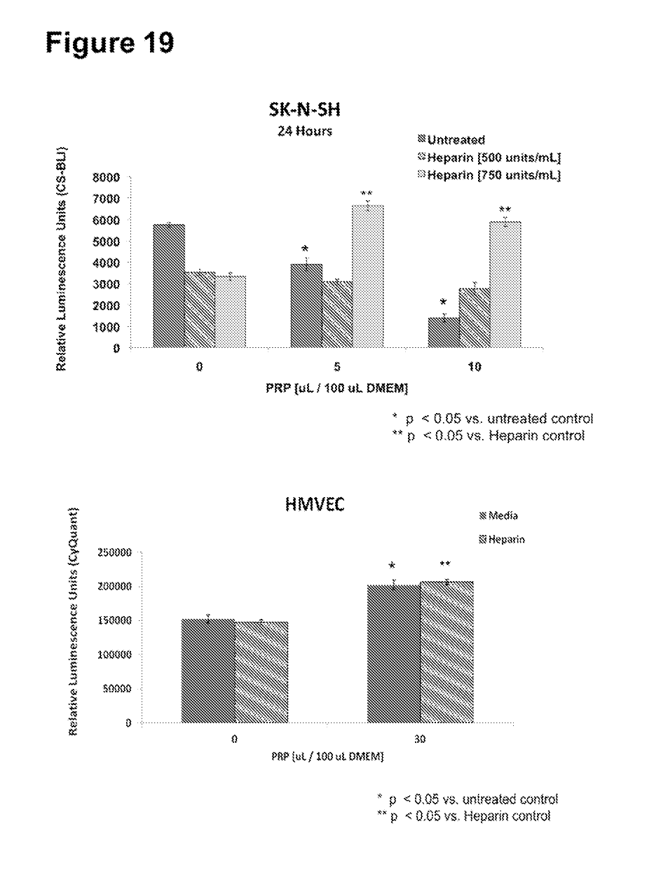

Additionally, platelets also mediated inhibitory effects on other tumor cell types, including MCF-7, MDA-231, and MDA-231-MET (each a human breast cancer cell line) and PC-3 (a human prostate cancer cell line) (FIG. 18). Consistent with the hypothesis that platelets regulate the sequestration and release of growth factors and chemokines through heparin binding in different microenvironments, heparin rescued SK-N-SH neuroblastoma cells from platelet-mediated apoptosis but did not alter platelet-mediated HMVEC proliferation (FIG. 19).

The data presented in Examples 3 and 4 indicate that platelets loaded with an active agent comprising a heparin binding domain can recognize a variety of tumor cells and specifically deliver their cargo to tumor cells. Depending on the active agent, e.g., an inhibitory compound, a chemotherapeutic agent, and peptide drug, this process could be useful inhibiting or killing a neoplastic cell.

It is expected that the processes demonstrated in Examples 3 and 4 would occur via interactions between a loaded platelet and the tumor cell and is independent of the cell's context, i.e., whether the cell is in vitro or in vivo.

### Example 5

### Platelets have Tumor Inhibitory Effects In Vivo.

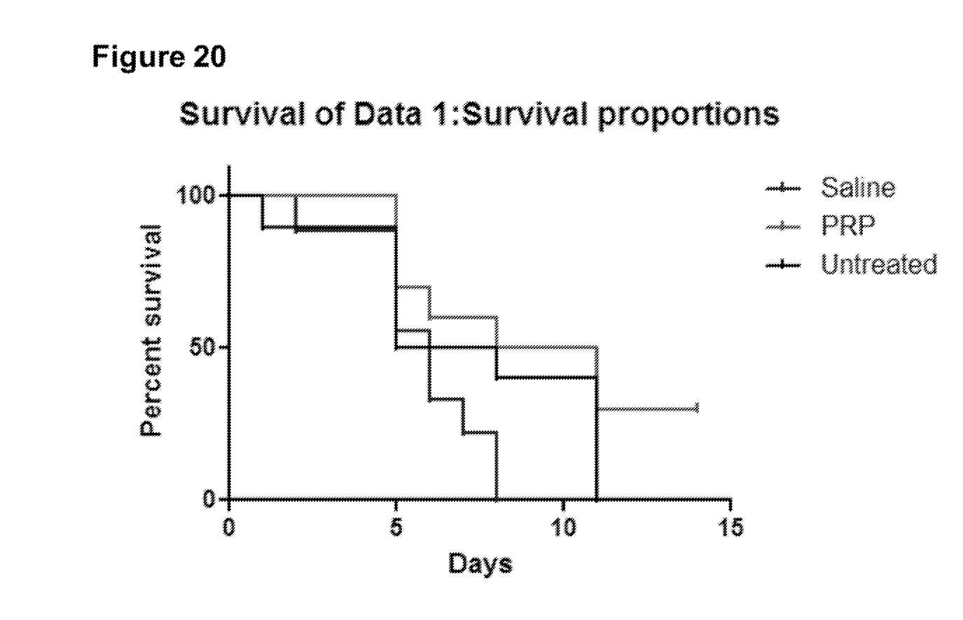

To test the tumor inhibitory effect of the platelets in vivo an orthotopic SK-N-SH neuroblastoma mouse model was used. Infusion of 200 μL of human PRP Q3 days improved the survival of mice with disseminated orthotopic SK-N-SH neuroblastoma (FIG. 20).

Tumors are communities of resident and migrated cells. Their growth is modulated by a dynamic reciprocity of it individual components—the host cells, stromal components and cancer cells. Invasion, progression and dissemination of a tumor are therefore a quorum effect of a wide number of inflammatory cells and stromal elements. As shown herein, platelets, the earliest responders to injury or tissue invasion, can mediate these interactions. Furthermore, platelets respond differently to different cells and recruit additional effector cells to wounds or tumor sites.

The outcome of platelet interaction with surrounding cells can be variable, and depends on the balance of growth stimulators and inhibitors. Under normal physiological conditions, the predominant effect of platelets is inhibition of growth. However, in the setting of vascular injury, cancer or other pathologic conditions, this balance is overturned, and the effect of platelets is growth stimulation. Platelets sequester angiogenesis regulators such as Vascular Endothelial Growth Factor (VEGF), basic Fibroblast Growth Factor (bFGF), Platelet Derived Growth Factor (PDGF), Platelet Factor 4 (PF4), and endostatin (ES) among others. This sequestration is active, against the concentration gradient in plasma, and highly selective, as platelets do not take up non-angiogenic proteins, even those that were very abundant in plasma.

While it was originally thought that the uptake of angiogenesis regulators is mediated by their respective receptors, evidence now exists to the contrary. A common mechanism involving heparin binding exists and accounts for the selectivity of angiogenesis regulators uptake by platelets, as well as for their sequential release. Thus, a major regulator of uptake and release of angiogenesis regulators are tissue proteoglycans. The affinity of individual proteins for heparin provides a mechanism for the spatially and temporarily orchestrated release of stimulators and inhibitors of angiogenesis (e.g., in wound healing, tumor growth, atherosclerosis and other angiogenesis dependent diseases). As platelets are recruited to sites of angiogenesis and inflammation, this provides the potential for the targeted delivery of agents for the treatment of disease at these sites.

These data indicate that the methods described above would be useful and effective in treating a subject having a tumor or neoplasia.

### Example 6

### The Present Invention can be Used in Variable Clinical Therapeutic Protocols

A significant advantage of the present invention is an ability of a clinician to modify a therapeutic protocol based factors related to a subject's disorder. Examples of these factors include but are not limited to his/her specific disorder, the severity of the disorder, the stage of the disorder, the responsiveness of the disorder to prior treatments, and so forth.

A clinician can administer to a subject an effective amount of a platelet of the present invention loaded with a one or more therapeutic compounds, or a composition comprising a singularly loaded platelet (having only one type of cargo), a composition comprising a mixture loaded platelet (having a variety of types of cargos), or a composition comprising multiple types of loaded platelets described herein to produce such effect. The amount of cargo loaded may be a small amount (i.e., the same amount as normally present or less than that normally present in a platelet) or a large amount (i.e., 2 to 1000 times as much as normally present in a platelet and much larger than possible using other known methods, e.g., liposome or tagged fusion proteins). The specific composition administered can vary among doses such that the amount of loaded platelets, the amount of cargo loaded in platelets, and the types of cargo(s) loaded can change. Thus, the combination of possible compositions and dosing regimens is vast.

A dosing regimen may approximate a naturally occurring process (e.g., wound healing as shown in FIG. 9, top). Alternately, it may vary from the natural process to obtain an un-natural outcome, yet desirable to the clinician and/or subject.

FIG. 21 provides examples of various loaded platelets each containing a different specific cargo, it provides examples of how a single type of loaded platelet or different types of loaded platelets can be used in a composition, and it provides examples of how a single composition can be used is a treatment does or a plurality of compositions can be combined into one treatment does. FIG. 21 additionally illustrates how a treatment regimen can vary over time and as the treatment progresses.

Certain results reported herein were obtained using the following methods and materials unless indicated otherwise.

### Sequential Release Experiments

To determine when various angiogenic proteins elute from a heparin column, platelets were isolated and incubated in a heparin column Proteins were eluted, and ELISA assays were used to quantify the proteins in each fraction.

Blood Collection and Isolation of Platelets: Healthy Human blood was collected into 5 mL citrated tubes and spun at 150 g (Eppendorf) for 30 min to isolate platelets. Platelet Rich Plasma (PRP) was removed (Ranin pipet) and placed in 1.5 Eppendorf tubes. PRP was rocked on a neutator (Fisher) until used.

Heparin Columns: Three, 1.0 mL Heparin Affinity Columns (GE Bioscience) were washed with 10 bead volumes of physiological saline (Hosperia). All solutions were added to the columns using a syringe and syringe adaptor with a 0.5 mL flow/min rate. 1 mL of isolated PRP was added to each column, and columns were incubated at 37 C for one hour in a rocking oven. Columns were then placed in tandem and washed twice with 10 column lengths (30 mL) of saline. NaCl (Sigma) solutions were premade using dilutions from high molar solutions in Milli-Q water. After washing of columns, 3 mL of NaCl solution was added to each column in sequential order. Elutions were collected in 15 mL (Corning) tubes and frozen at −80 C until ELISA assay were performed.

ELISA assays: ELISA assays from R and D Systems, following standard ELISA protocol for each individual protein, were used to quantify protein in each elution. ELISA assays were read at 450 nm using Victor™ 3 plate reader (Perkin Elmer). An additional reading was done at 562 nm, in order to obtain a background value. In order to determine the maximal regression curve, only standard protein values in the dynamic range of the experiment were used. Due to regression curve, any negative values were adjusted to 0.

### Methods for CS-BLI Experiments (Cell Specific Bio-Luminescence Imaging)

To determine the viability of SK-N-SH tumor cells without interference due to platelet addition, the CS-BLI technique was adopted, (McMillin et al 2010 Nature Medicine). SK-N-SH cells (Neuroblastoma, ATCC®) expressing luciferase were cultured in 96 well luminescence plates (Nuncleon Delta plates, Thermo) at a density of 3000 or 5000 cells/well for 24 hours in order to attach to the plates. Cells were then treated with varying concentrations of PRP and/or drugs and incubated for 24 hours. Each condition was run in octuplet. Thirty minutes prior to measurement 10 uL of D-Luciferin (Caliper) was added to each well with a multi-channel pipet (Ranin) at a concentration of 2.5 mg/mL and incubated at 37 C. Plates were then measured using Victor 3 (Perkin Elmer) multi-channel plate reader. Data was exported and analyzed in Excel (Microsoft®)

### Methods for Measurement of Cell Proliferation:

To determine the proliferation of HMVEC or SKNSH cells due to platelets or protein treatments, CyQuant® (Invitrogen) proliferation assays were used. HMVEC or SK-N-SH cells (ATCC®) were grown on 96 well fluorescence plates (Corning) at a density of 3000 or 5000 cells/well for 24 hours to adhere prior to the experiment. Once cells had adhered, cells were treated with PRP or growth factors (R and D Systems) at various concentrations in octuplet and incubated for 24 hours. The plates were then frozen at −80 overnight. The plates were then allowed to thaw for one hour at room temperature prior to addition of CyQuant® dye as described by the manufacturer and measured using Victor 3 (Perkin Elmer) multi-channel plate reader. Data was exported and analyzed in Excel (Microsoft)

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for delivering an agent to a site of injury, inflammation, disease, or disorder in a subject in need thereof, the method comprising:
loading one or more samples of platelets with one or more agents, each agent comprising a heparin binding domain, thereby producing one or more pools of loaded platelets;
administering a composition comprising one or more pools of loaded platelets to the subject in need thereof,
wherein an individual loaded platelet present in the composition localizes to a site of injury, inflammation, disease, or disorder and delivers its one or more agents to the site of injury, inflammation, disease, or disorder.

2. The method of claim 1, wherein the method comprises administering a plurality of sequential doses of compositions.

3. The method of claim 2, wherein the plurality of sequential doses of compositions comprises compositions each including platelets loaded with identical one or more agents.

4. The method of claim 3, wherein loaded platelets in a composition include a lesser amount, the same amount, or a greater amount of the identical one or more agents than the amount loaded into platelets in another composition.

5. The method of claim 2, wherein the plurality of sequential doses of compositions comprises a variety of compositions.

6. The method of claim 5, wherein loaded platelets in a composition include identical one or more agents or different one or more agents than agents loaded into platelets in another composition.

7. The method of claim 6, wherein when loaded platelets in a composition include identical one or more agents than agents loaded in another composition, the loaded platelets in the composition include a lesser amount, the same amount, or a greater amount of the identical one or more agents than the amount loaded into platelets in the other composition.

8. The method of any one of claims 1 to 7, wherein a specific composition administered to the subject is selected based upon the particular injury, inflammation, disease, or disorder afflicting the subject.

9. The method of any one of claims 1 to 8, wherein a specific composition administered to the subject is selected based upon the stage or severity of the particular injury, inflammation, disease, or disorder afflicting the subject.

10. The method of any one of claims 1 to 9, wherein a specific composition administered to the subject is selected based upon characteristics of the subject including but not limited to the subject's response to a previously administered composition if a composition had been previously administered.

11. The method of any one of claims 1 to 10, the method further comprising administering heparinase, a PAR1 agonist peptide, and/or a PAR4 agonist peptide to enhance release of the one or more agents from the loaded platelet.

12. The method of any one of claims 1 to 11, wherein at least one sample of platelets comprises autologous platelets.

13. The method of any one of claims 1 to 12, wherein the injury, inflammation, disease, or disorder is selected from the group consisting of a wound, tumor, atherosclerotic plaque, macular degeneration, skin proliferation/ulceration (psoriasis, rosacea), gastrointestinal proliferation/ulceration (ulcerative colitis, Crohn's disease), arthritis and joint disease (synovial plague), endometriosis, site of neural degeneration (Alzheimer Disease, ALS, MS), post infectious angiogenesis (Bartonella, shigelosis, cerebral malaria), embryo implantation, preeclampsia, obesity, neuropathy, and tissue regeneration.

14. The method of any one of claims 1 to 13, wherein the one or more agents is a recombinant fusion polypeptide.

15. The method of claim 14, wherein the heparin binding domain is operably linked to the recombinant fusion polypeptide.

16. The method of any one of claims 1 to 15, wherein the one or more agents is a growth factor, a growth inhibitor, a protease/proteinase, a coagulation factor, a lipid or phospholipid, an extracellular matrix protein, a hormone, an enzyme, a chemokine/chemoattractant, or a neurotrophin.

17. The method of claim 16, wherein the growth factor is selected from the group consisting of vascular endothelial growth factor (VEGF), basic fibroblast growth factor (bFGF), and platelet-derived growth factor (PDGF), Epidermal Growth Factor (EGF), Hepatocyte Growth Factor (HGF), Insulin-Like Growth Factor (IGF), and an Angiopoietin.

18. The method of claim 16, wherein the growth inhibitor is selected from the group consisting of angiostatin, endostatin, tumstatin, Thrombospondin-1 (TSP1), Platelet Factor 4 (PF4, CXCL4), and Tissue Inhibitors of Metalloproteinases (TIMPs).

19. The method of claim 16, wherein the protease/proteinase is selected from the group consisting of Matrix Metalloproteinases (MMPs), thrombin, tissue plasminogen activator (tPA), urokinase, and streptokinase.

20. The method of claim 16, wherein the coagulation factor is selected from the group consisting of Factor II (thrombin), Antithrombin III (ATIII), Kallikrein, tissue factor (TF), Factor V, Factor VII, Factor VIII, Factor IX, Factor X, Factor XI, and Factor XII, Factor XIII, Fibrinogen, Protein S, Protein C, thrombomodulin, plasminogen, and tissue factor pathway inhibitor (TFPI).

21. The method of claim 16, wherein the lipid or phospholipid is selected from the group consisting of apolipoprotein E (ApoE), platelet phospholipids, and Sphingosine-1-phosphate (S1P).

22. The method of claim 16, wherein the extracellular matrix protein is selected from the group consisting of integrins, fibronectin, laminin, focal adhesion proteins (FAK), vinculin, talin, actin filaments, and collagen.

23. The method of claim 16, wherein the hormone is selected from the group consisting of insulin, steroid, erythropoietin, thrombopoietin, and thyroid hormone.

24. The method of claim 16, wherein the enzyme is Heparanase or a Matrix Metalloproteinase (MMP).

25. The method of claim 16, wherein the chemokine/chemoattractant is selected from the group consisting of Connective Tissue Growth Factor (CTGF), Stromal Cell-derived Factor-1 ( SDF-1) (CXCL12), interleukins (IL1, 2, 6, 8), and CD40 Ligand (CD40L, CD154).

26. The method of claim 16, wherein the neurotrophin is selected from the group consisting of nerve growth factor (NGF), brain-derived neurotrophic factor (BDNF), Neurotrophin-3 (NT-3), and Neurotrophin 4/5 (NT-4/5).

27. The method of any one of claims 1 to 15, wherein the one or more agents is a cytotoxic compound, a small molecule, an antibody, or a factor that inhibits angiogenesis.

28. The method of any one of claims 1 to 27, wherein the one or more samples of platelets is obtained from Platelet Rich Plasma (PRP).

29. The method of any one of claims 1 to 28, wherein a loaded platelet comprises 1 to 1000 fold more copies of the one or more agents than the platelet comprised prior to loading the one or more agents.

30. The method of claim 29, wherein the loaded platelet comprises up to 600 fold more copies of the one or more agents than the platelet comprised prior to loading the one or more agents.

31. A method for preparing a platelet loaded with one or more agents, the method comprising
obtaining a platelet,
contacting the platelet in vitro with the one or more agents, each agent comprising a heparin binding domain,
allowing contact between the platelet and the one or more agents to progress until the one or more agents is internalized by the platelet, thereby producing a loaded platelet.

32. The method of claim 31, wherein the platelet is an autologous platelet.

33. The method of claim 31 or claim 32, wherein the one or more agents is a recombinant fusion polypeptide.

34. The method of claim 33, wherein the heparin binding domain is operably linked to the recombinant fusion polypeptide.

35. The method of any one of claims 31 to 34, wherein the one or more agents is a growth factor, a growth inhibitor, a protease/proteinase, a coagulation factor, a lipid or phospholipid, an extracellular matrix protein, a hormone, an enzyme, a chemokine/chemoattractant, or a neurotrophin.

36. The method of claim 35, wherein the growth factor is selected from the group consisting of vascular endothelial growth factor (VEGF), basic fibroblast growth factor (bFGF), and platelet-derived growth factor (PDGF), Epidermal Growth Factor (EGF), Hepatocyte Growth Factor (HGF), Insulin-Like Growth Factor (IGF), and an Angiopoietin.

37. The method of claim 35, wherein the growth inhibitor is selected from the group consisting of angiostatin, endostatin, tumstatin, Thrombospondin-1 (TSP1), Platelet Factor 4 (PF4, CXCL4), and Tissue Inhibitors of Metalloproteinases (TIMPs).

38. The method of claim 35, wherein the protease/proteinase is selected from the group consisting of Matrix Metalloproteinases (MMPs), thrombin, tissue plasminogen activator (tPA), urokinase, and streptokinase.

39. The method of claims 35, wherein the coagulation factor is selected from the group consisting of Factor II (thrombin), Antithrombin III (ATIII), Kallikrein, tissue factor (TF), Factor V, Factor VII, Factor VIII, Factor IX, Factor X, Factor XI, and Factor XII, Factor XIII, Fibrinogen, Protein S, Protein C, thrombomodulin, plasminogen, and tissue factor pathway inhibitor (TFPI).

40. The method of claim 35, wherein the lipid or phospholipid is selected from the group consisting of apolipoprotein E (ApoE), platelet phospholipids, and Sphingosine-1-phosphate (S1P).

41. The method of claim 35, wherein the extracellular matrix protein is selected from the group consisting of integrins, fibronectin, laminin, focal adhesion proteins (FAK), vinculin, talin, actin filaments, and collagen.

42. The method of claim 35, wherein the hormone is selected from the group consisting of insulin, steroid, erythropoietin, thrombopoietin, and thyroid hormone.

43. The method of claim 35, wherein the enzyme is Heparanase or a Matrix Metalloproteinase (MMP).

44. The method of claim 35, wherein the chemokine/chemoattractant is selected from the group consisting of Connective Tissue Growth Factor (CTGF), Stromal Cell-derived Factor-1 (SDF-1) (CXCL12), interleukins (IL1, 2, 6, 8), and CD40 Ligand (CD40L, CD154).

45. The method of claim 35, wherein the neurotrophin is selected from the group consisting of nerve growth factor (NGF), brain-derived neurotrophic factor (BDNF), Neurotrophin-3 (NT-3), and Neurotrophin 4/5 (NT-4/5).

46. The method of any one of claims 31 to 34, wherein the one or more agents is a cytotoxic compound, a small molecule, an antibody, or a factor that inhibits angiogenesis.

47. The method of any one of claims 31 to 46, wherein the one or more samples of platelets is obtained from Platelet Rich Plasma (PRP).

48. The method of any one of claims 31 to 47, wherein a loaded platelet comprises 1 to 1000 fold more copies of the one or more agents than the platelet comprised prior to loading the one or more agents.

49. The method of claim 48, wherein the loaded platelet comprises up to 600 fold more copies of the one or more agents than the platelet comprised prior to loading the one or more agents.

50. An isolated platelet loaded with the one or more agents according to the method of any one of claims 31 to 49.

51. A composition comprising the loaded platelet claim 50.

52. A pharmaceutical composition comprising an effective amount of the loaded platelet of claim 50 in a pharmaceutically acceptable excipient.

53. A kit for treating an injury, inflammation, disease, or disorder, the kit comprising the loaded platelet, composition, or pharmaceutical composition of any one of claims 50 to 52.

54. The kit of claim 53, wherein the kit further comprises written instructions for using the platelet composition, or pharmaceutical composition in the treatment of a subject.

55. A kit for preparing a loaded platelet of claim 50.

56. The kit of claim 55, wherein the kit further comprises written instructions for preparing the loaded platelet and for uses thereof.
